(19) United States
(12) Patent Application Publication
(10) Pub. No.: US 2017/0268014 A1
(43) Pub. Date: Sep. 21, 2017

(54) REGULATORY NUCLEIC ACIDS AND METHODS OF USE

(71) Applicants: Syngenta Participations AG, Basel (CH); SYNGENTA CROP PROTECTION, LLC, Research Triangle Park, NC (US)

(72) Inventor: Michael L. NUCCIO, Research Triangle Park, NC (US)

(73) Assignee: Syngenta Participations AG, Basel (CH)

(21) Appl. No.: 15/329,103

(22) PCT Filed: Dec. 8, 2015
(86) PCT No.: PCT/US15/64423

§ 371 Date: Jan. 25, 2017

Related U.S. Application Data
(60) Provisional application No. 62/090,425, filed on Dec. 11, 2014.

(51) Int. Cl. C12N 15/82

(52) U.S. Cl. CPC C12N 15/8237; C12N 15/8279; C12N 15/8274; C12N 15/8242; C12N 15/8271; C12N 15/8261

(57) ABSTRACT

The present invention relates generally to the field of molecular biology and describes nucleic acids encoding regulatory elements capable of affecting expression of a coding sequence. The regulatory elements described herein may be used to direct the expression of a heterologous coding region in the green tissues and upon exposure to light in plants. The invention may also be used to create transgenic plants having improved characteristics, such as yield.

### RELATED APPLICATION INFORMATION

This Application claims the benefit of U.S. Provisional Application No. 62/090,425, filed 11 Dec. 2014, the contents of which are incorporated herein by reference.

### STATEMENT REGARDING ELECTRONIC FILING OF A SEQUENCE LISTING

A Sequence Listing in ASCII text format, submitted under 37 C.F.R. §1.821, entitled 80195-WO-REG-ORG-P-1_Sequence_Listing_ST25, 44 kilobytes in size, generated on Dec. 1, 2015 and filed via EFS-Web, is provided in lieu of a paper copy. This Sequence Listing is hereby incorporated by reference into the specification for its disclosures.

### FIELD OF THE INVENTION

The present invention relates to the fields of agriculture, plant breeding or genetic engineering for plants.

### BACKGROUND

A critical component of plant biotechnology is the use of promoters with unique spatial and temporal activity profiles to express agronomically important genes in crop plants so that genes of interest are expressed at optimal levels in appropriate tissues. In agricultural biotechnology, plants can be modified according to one's needs. One way to accomplish this is by using modern genetic engineering techniques. For example, by introducing a gene of interest into a plant, the plant can be specifically modified to express a desirable phenotypic trait. For this, plants are transformed most commonly with a heterologous gene comprising a promoter region, a coding region and a termination region. When genetically engineering a heterologous gene for expression in plants, the selection of a promoter is often a factor. While it can be desirable to express certain genes constitutively, i.e. throughout the plant at all times and in most tissues and organs, other genes are more desirably expressed only in response to particular stimuli or confined to specific cells or tissues.

### SUMMARY OF THE INVENTION

One embodiment of the invention is a nonnaturally occurring light inducible regulatory nucleic acid comprising a regulatory nucleic acid having at least 90 percent or greater sequence identity to a nucleic acid selected from the group consisting of SEQ ID NO: 9, 10, 11 and 12; or a regulatory nucleic acid selected from the group consisting of SEQ ID NO: 9, 10, 11 and 12 or a functionally equivalent fragment thereof; or a regulatory nucleic acid selected from the group consisting of SEQ ID NO: 9, 10, 11 and 12; wherein said regulatory nucleic acid directs transcription of an operably linked polynucleotide in a plant.

The nucleic acid may be a functionally equivalent fragment comprising at least 200, 300 or 400 base pairs of SEQ ID NO: 9, 10, 11 or 12. In some embodiments the nucleic acid may be operably linked to an intron. In addition, the nucleic acid may be operably linked to a terminator. In one embodiment, the promoter, intron and terminator are isolated from the same gene or coding region. Alternatively, the promoter, intron and terminator may be isolated from more than one gene or coding region.

Another embodiment is an expression cassette comprising a first nucleic acid, wherein the first nucleic acid is a nonnaturally occurring light inducible regulatory nucleic acid comprising a regulatory nucleic acid having at least 90 percent or greater sequence identity to a nucleic acid selected from the group consisting of SEQ ID NO: 9, 10, 11 and 12; or a regulatory nucleic acid selected from the group consisting of SEQ ID NO: 9, 10, 11 and 12 or a functionally equivalent fragment thereof; or a regulatory nucleic acid selected from the group consisting of SEQ ID NO: 9, 10, 11 and 12; wherein said regulatory nucleic acid directs transcription of an operably linked polynucleotide in a plant.; a second nucleic acid to be transcribed, wherein said first and second nucleic acids are heterologous to each other and are operably linked; and a terminator operably linked 3′ to the nucleic acid to be transcribed. The first and second nucleic acids are heterologous to each other and are operably linked; and a terminator operably linked 3′ to the nucleic acid to be transcribed. The second nucleic acid may be selected from the group comprising a pest resistance nucleic acid, a disease resistance nucleic acid, an herbicide resistance nucleic acid, a value-added trait nucleic acid, a photoassimilation nucleic acid, a yield nucleic acid and a stress tolerance nucleic acid. In addition, the heterologous coding region may be green tissue and/or light regulated, such that, transcription of the coding region is promoted, induced or active in the presence of light.

In another embodiment, a plant, plant tissue, or plant cell comprising any of the above described expression cassettes. The plant, plant tissue, or plant cell can be a monocot or from monocot, such as, maize or a dicot, such as soybean.

Another embodiment is a method of expressing a heterologous coding region comprising a regulatory nucleic acid having at least 90 percent, 95 percent, 98 percent or greater sequence identity to the nucleotide sequences set forth in SEQ ID NO: 9, 10, 11 or 12; a regulatory nucleic acid comprising a nucleotide sequence set forth in SEQ ID NO: 9, 10, 11 or 12 or a functionally equivalent fragment thereof; or a regulatory nucleic acid comprising a nucleotide sequence set forth in SEQ ID NO: 9, 10, 11 or 12 operably linked to a heterologous coding region; and creating a plant, plant tissue, or plant cell comprising the nucleic acid, wherein the heterologous coding region is expressed. The heterologous coding region may be expressed in green tissue and/or light regulated such that, transcription of the coding region is promoted, induced or active in the presence of light. The plant, plant tissue, plant cell or a portion thereof may be a monocot, from a monocot, such as, maize or a dicot, from a dicot, such as, soybean.

Another embodiment includes a plant, plant tissue, plant cell, or portion thereof made by the method of expressing a heterologous coding region comprising providing a regulatory nucleic acid having at least 90 percent, 95 percent, 98 percent or greater sequence identity to the nucleotide sequences set forth in SEQ ID NO: 9, 10, 11 or 12; a regulatory nucleic acid comprising a nucleotide sequence set forth in SEQ ID NO: 9, 10, 11 or 12 or a functionally equivalent fragment thereof; or a regulatory nucleic acid comprising a nucleotide sequence set forth in SEQ ID NO: 9, 10, 11 or 12 operably linked to a heterologous coding region; and creating a plant, plant tissue, or plant cell comprising the nucleic acid, wherein the heterologous coding region is expressed. Included is the progeny, seed, or grain produced by the plant, plant tissue, plant cell, or portion thereof.

Additionally, an embodiment may be the use of a nonnaturally occurring nucleic acid to promote expression of a heterologous transgene in the presence of light, wherein the nucleic acid is selected from a group comprising SEQ ID NO: 9, 10, 11 and 12.

### BRIEF DESCRIPTION OF THE SEQUENCES IN THE SEQUENCE LISTING

SEQ ID NO: 1 is fructose-bisphosphate aldolase, chloroplast precursor (ALDP) from rice, polynucleotide sequence.

SEQ ID NO: 2 is fructose-bisphosphate aldolase from rice, polypeptide.

SEQ ID NO: 3 is Sedoheptulose-1,7-bisphosphatase from rice, polynucleotide.

SEQ ID NO: 4 is Sedoheptulose-1,7-bisphosphatase from rice, polypeptide.

SEQ ID NO: 5 is ADP-glucose pyrophosphorylase from rice, AGPS2a small subunit, polynucleotide.

SEQ ID NO: 6 is ADP-glucose pyrophosphorylase from rice, AGPS2a small subunit, polypeptide.

SEQ ID NO: 7 is ADP-glucose pyrophosphorylase from rice, AGP13, large subunit, polynucleotide.

SEQ ID NO: 8 is ADP-glucose pyrophosphorylase from rice, AGP13, large subunit, polypeptide.

SEQ ID NO: 9 is OsLHC3 promoter from rice, polynucleotide.

SEQ ID NO: 10 is OsLHC4 promoter from rice, polynucleotide.

SEQ ID NO: 11 is OsPsak promoter from rice, polynucleotide.

SEQ ID NO: 12 is OsPSID promoter from rice, polynucleotide.

SEQ ID NO: 13 is OsLHCA3, first exon of the OsLHCA3 gene from rice, polynucleotide.

SEQ ID NO: 14 is OsLHCA3, first intron of the OsLHCA3 gene from rice, polynucleotide

SEQ ID NO: 15 is OsLHCA3, second exon of the OsLHCA3 gene from rice, polynucleotide

SEQ ID NO: 16 is OsLHCA3, second intron of the OsLHCA3 gene from rice, polynucleotide

SEQ ID NO: 17 is OsLHCA3 terminator, polynucleotide

SEQ ID NO: 18 is TMV-Ω tobacco mosaic translation enhancer fused to a soy-optimized Kozak sequence.

SEQ ID NO: 19 is OsLHC4 first exon from rice, polynucleotide.

SEQ ID NO: 20 is OsLHC4 first intron from rice, polynucleotide.

SEQ ID NO: 21 is OsLHC4 terminator from rice, polynucleotide.

SEQ ID NO: 22 is TMV-07 tobacco mosaic virus enhancer fused to a soy-optimized Kozak sequence.

SEQ ID NO: 23 is OsPsak first exon from rice, polynucleotide.

SEQ ID NO: 24 is OsPsak first intron from rice, polynucleotide.

SEQ ID NO: 25 is OsPsak terminator from rice, polynucleotide.

SEQ ID NO: 26 is NtADH translational enhancer based on the tobacco alcohol dehydrogenase gene sequence with soy optimized Kozak sequence.

SEQ ID NO: 27 is OsPSID terminator from rice.

SEQ ID NO: 28 is TMV-omega translational enhancer complex, M14 version with a soy-optimized Kozak sequence.

### DETAILED DESCRIPTION OF THE INVENTION

It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

A promoter is a region which facilitates the transcription of a specific gene or coding region. Transcription factors bind to promoter regions at specific sequences. Binding motifs for transcription factors can be predicted in promoter sequence. Some motifs are annotated as light inducible, i.e. transcription of the gene or coding region occurs upon exposure to light. In some embodiments of the invention, the promoters described contain one or more motifs selected from the group consisting of a BOXIIPCCHS motif, CIACADIANLELHC motif, GT1CONSENSUS motif, IBOX motif, IBOXCORE motif, IBOXCORENT motif, INRNTPSADB motif, LRENPCABE motif, SORLIP1AT motif, SORLIP2AT and SORLIP5AT motif.

The promoter, intron and terminator sequences and methods of use disclosed herein may be used in combination with any one of the following elements such as enhancers, upstream elements, and/or activating sequences from the 5′ flanking regions of plant expressible structural genes. In some embodiments of this invention, the regulatory nucleic acids comprise a promoter, a first exon, an intron, and optionally a second exon or fragment thereof. Alternatively, the regulatory nucleic acids may combine a promoter, intron and terminator. These regulatory nucleic acids may or may not be derived from the same locus of a plant genome. The regulatory nucleic acids may comprise the first or 5′ most exon of the locus, the 5′ most intron and the second exon immediately downstream of the 5′ most intron in the genome of the non-transgenic plant. For example, please see U.S. Pat. No. 8,129,588; which is hereby incorporated by reference.

By “host cell” is meant a cell, which comprises a heterologous nucleic acid sequence of the invention, which contains a vector and supports the replication and/or expression of the vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to sunflower, soybean, tobacco, wheat, alfalfa, rice, cotton, rapeseed, spinach, sugar beet, Arabidopsis and tomato. A particularly preferred dicotyledonous host cell is a soybean host cell and a particularly preferred monocotyledonous host cell is a maize host cell.

In some embodiments, the invention provides an expression cassette that may be used to drive expression of heterologous genes or heterologous coding regions for increasing yield, or improving resistance to herbicides, pests, disease or drought. Some embodiments provide expression cassettes to express heterologous or chimeric genes or coding regions in response to light. This expression may occur in green tissues such as leaves.

“Expression cassette” as used herein means a nucleic acid molecule capable of directing expression of a particular polynucleotide or polynucleotides in an appropriate host cell, comprising a promoter operably linked to the polynucleotide or polynucleotides of interest which is/are operably linked to a terminator. It also typically comprises polynucleotides required for proper translation of the polynucleotide or polynucleotides of interest. The expression cassette may also comprise polynucleotides not necessary in the direct expression of a polynucleotide of interest but which are present due to convenient restriction sites for removal of the cassette from an expression vector. The expression cassette comprising the polynucleotide(s) of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e. the particular polynucleotide of the expression cassette does not occur naturally in the host cell and must have been introduced into the host cell or an ancestor of the host cell by a transformation process known in the art. The expression of the polynucleotide(s) in the expression cassette is generally under the control of a promoter. In the case of a multicellular organism, such as a plant, the promoter can also be specific or preferential to a particular tissue, or organ, or stage of development. An expression cassette, or fragment thereof, can also be referred to as “inserted polynucleotide” or “insertion polynucleotide” when transformed into a plant.

The expression cassettes may be introduced in to host cells, including plant cells. The plant cell may be regenerated into a plant comprising the expression cassettes. For example, the plant may be a monocot or dicot plant. In some embodiments, the plant is selected from the group consisting of maize, sugarcane, sorghum, amaranth, rice, soybean, wheat, tobacco, sugar beet, sunflower, rapeseed, and Arabidopsis. In some embodiments the plant is a maize or soybean plant.

Additional embodiments of the invention include methods of producing a transgenic plant or methods of increasing yield in a plant comprising introducing one of the expression cassettes of the invention into a plant and producing or regenerating a transgenic plant. The transgenic plant may be crossed with a non-transgenic plant and then selected for a progeny plant comprising one of the expression cassettes of the invention.

It is to be understood that this invention is not limited to the particular methodology, protocol, cell line, plant species or genera, constructs, and reagents described herein as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a plant” is a reference to one or more plants and includes equivalents thereof known to those skilled in the art, and so forth. As used herein, the word “or” means any one member of a particular list and also includes any combination of members of that list (i.e., includes also “and”).

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this invention (e.g., in combinations with temperature or molecular weight values) the exact value (i.e., without “about”) is preferred.

“Antisense inhibition” refers to the production of antisense RNA transcripts capable of suppressing the expression of protein from an endogenous gene or a transgene.

“Cis-element” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact with different affinities with more than one cis-element. Cis-elements can be identified by a number of techniques, including deletion analysis, i.e., deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis with known cis-element motifs by conventional DNA sequence comparison methods. The fine structure of a cis-element can be further studied by mutagenesis (or substitution) of one or more nucleotides or by other conventional methods. Cis-elements can be obtained by chemical synthesis or by isolation from promoters that include such elements, and they can be synthesized with additional flanking nucleotides that contain useful restriction enzyme sites to facilitate subsequence manipulation.

The term “chimeric construct”, “chimeric gene”, “chimeric polynucleotide” or chimeric nucleic acid” (and similar terms) as used herein refers to a construct or molecule comprising two or more polynucleotides of different origin assembled into a single nucleic acid molecule. The term “chimeric construct”, “chimeric gene”, “chimeric polynucleotide” or “chimeric nucleic acid” refers to any construct or molecule that contains (1) polynucleotides (e.g., DNA), including regulatory and coding polynucleotides that are not found together in nature (i.e., at least one of polynucleotides is heterologous with respect to at least one of its other polynucleotides), or (2) polynucleotides encoding parts of proteins not naturally adjoined, or (3) parts of promoters that are not naturally adjoined. Further, a chimeric construct, chimeric gene, chimeric polynucleotide or chimeric nucleic acid may comprise regulatory polynucleotides and coding polynucleotides that are derived from different sources, or comprise regulatory polynucleotides and coding polynucleotides derived from the same source, but arranged in a manner different from that found in nature. In a preferred aspect of the present invention the chimeric construct, chimeric gene, chimeric polynucleotide or chimeric nucleic acid comprises an expression cassette comprising a polynucleotides of the present invention.

“Chromosomally-integrated” refers to the integration of a foreign gene or DNA construct into the host DNA by covalent bonds. Where genes or coding regions are not “chromosomally integrated”, they may be “transiently expressed.” Transient expression of a gene or coding region refers to the expression of a gene or coding region that is not integrated into the host chromosome but functions independently, either as part of an autonomously replicating plasmid or expression cassette, for example, or as part of another biological system such as a virus.

A “coding region” or “coding region polynucleotide” is a polynucleotide that is transcribed into RNA, such as mRNA, rRNA, tRNA, snRNA, sense RNA or antisense RNA. Preferably the RNA is then translated in an organism to produce a protein. It may constitute an “uninterrupted coding polynucleotide”, i.e., lacking an intron, such as in a cDNA, or it may include one or more introns bounded by appropriate splice junctions. An “intron” is a poly(ribo)nucleotide which is contained in the primary transcript but which is removed through cleavage and religation of the RNA within the cell to create the mature mRNA that can be translated into a protein.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

“Contiguous” is used herein to mean nucleic acid sequences that are immediately preceding or following one another.

“dsRNA” or “double-stranded RNA” is RNA with two substantially complementary strands, which directs the sequence-specific degradation of mRNA through a process known as RNA interference (RNAi). dsRNA is cut into siRNAs interfering with the expression of a specific gene.

The term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In some embodiments, “expression” refers to the transcription and stable accumulation of sense (mRNA) or functional RNA. “Expression” may also refer to the production of protein.

A “gene” is defined herein as a hereditary unit consisting of a polynucleotide that occupies a specific location on a chromosome and that contains the genetic instruction for a particular characteristic or trait in an organism.

“Genetic engineering”, “transformation” and “genetic modification” are all used herein as synonyms for the transfer of isolated, nonnaturally occurring or synthetic genes into the DNA, usually the chromosomal DNA or genome, of another organism.

The term “genotype” refers to the genetic constitution of a cell or organism. An individual's “genotype for a set of genetic markers” includes the specific alleles, for one or more genetic marker loci, present in the individual. As is known in the art, a genotype can relate to a single locus or to multiple loci, whether the loci are related or unrelated and/or are linked or unlinked. In some embodiments, an individual's genotype relates to one or more genes that are related in that one or more of the genes are involved in the expression of a phenotype of interest (e.g., a quantitative trait as defined herein). Thus, in some embodiments a genotype comprises a sum of one or more alleles present within an individual at one or more genetic loci of a quantitative trait. In some embodiments, a genotype is expressed in terms of a haplotype (defined herein below).

The term “heterologous” when used in reference to a gene or nucleic acid refers to a gene encoding a factor that is not in its natural environment (i.e., has been altered by the hand of man). For example, a heterologous gene or heterologous coding region may include a gene or coding region from one species introduced into another species. A heterologous coding region may also include a coding region native to an organism that has been altered in some way (e.g., mutated, added in multiple copies, linked to a non-native promoter or enhancer polynucleotide, etc.). Heterologous coding regions further may comprise plant polynucleotides that comprise cDNA forms of a protein coding region; the cDNAs may be expressed in either a sense (to produce mRNA) or anti-sense orientation (to produce an anti-sense RNA transcript that is complementary to the mRNA transcript). In one aspect of the invention, heterologous coding regions are distinguished from endogenous plant coding regions in that the heterologous coding region polynucleotides are typically joined to polynucleotides comprising regulatory elements such as promoters that are not found naturally associated with the gene for the protein encoded by the heterologous coding region or with a plant coding region polynucleotide in the chromosome, or are associated with portions of the chromosome not found in nature (e.g., genes expressed in loci where the gene is not normally expressed). Further, in embodiments, a “heterologous” polynucleotide is a polynucleotide not naturally associated with a host cell into which it is introduced, including non-naturally occurring multiple copies of a naturally occurring polynucleotide.

The terms “homology”, “sequence similarity” or “sequence identity” of nucleotide or amino acid sequences mean a degree of identity or similarity of two or more sequences and may be determined conventionally by using known software or computer programs such as the Best-Fit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2:482-489 (1981), to find the best segment of identity or similarity between two sequences. Sequence comparison between two or more polynucleotides or polypeptides is generally performed by comparing portions of the two sequences over a comparison window to identify and compare local regions of sequence similarity. The comparison window is generally from about 20 to 200 contiguous nucleotides. Gap performs global alignments: all of one sequence with all of another similar sequence using the method of Needleman and Wunsch, J. Mol. Biol. 48:443-453 (1970). When using a sequence alignment program such as BestFit to determine the degree of DNA sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The term “isolated” or “nonnaturally occurring”, when used in the context of the nucleic acid molecules or polynucleotides of the present invention, refers to a polynucleotide that is identified within and nonnaturally occurring/separated from its chromosomal polynucleotide context within the respective source organism. An nonnaturally occurring nucleic acid or polynucleotide is not a nucleic acid as it occurs in its natural context, if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The nonnaturally occurring nucleic acid molecule may be present in single-stranded or double-stranded form. Alternatively, it may contain both the sense and antisense strands (i.e., the nucleic acid molecule may be double-stranded). In a preferred embodiment, the nucleic acid molecules of the present invention are understood to be nonnaturally occurring.

“5′ non-coding sequence” refers to a nucleotide sequence located 5′ (upstream) to the coding sequence. It is present in the fully processed mRNA upstream of the translation initiation codon and may affect processing of the primary transcript to mRNA, mRNA stability or translation efficiency. (Turner et al., 1995, Molecular Biotechnology, 3:225).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989, Plant Cell, 1:671-680).

The phrase “nucleic acid” or “polynucleotide” refers to any physical string of monomer units that can be corresponded to a string of nucleotides, including a polymer of nucleotides (e.g., a typical DNA polymer or polydeoxyribonucleotide or RNA polymer or polyribonucleotide), modified oligonucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Operably linked” refers to the association of polynucleotides on a single nucleic acid fragment so that the function of one affects the function of the other. For example, a promoter is operably linked with a coding polynucleotide or functional RNA when it is capable of affecting the expression of that coding polynucleotide or functional RNA (i.e., that the coding polynucleotide or functional RNA is under the transcriptional control of the promoter). Coding polynucleotide in sense or antisense orientation can be operably linked to regulatory polynucleotides.

“Overexpression” refers to the level of expression in transgenic organisms that exceeds levels of expression in normal or untransformed organisms.

“Primary transformant” and “TO generation” refer to transgenic plants that are of the same genetic generation as the tissue that was initially transformed (i.e., not having gone through meiosis and fertilization since transformation). “Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The terms “protein,” “peptide” and “polypeptide” are used interchangeably herein.

“Promoter” refers to a nucleic acid, which controls the expression of a coding sequence or gene by providing the recognition for RNA polymerase and other factors required for proper transcription. “Promoter regulatory sequences” or “promoter regulatory nucleic acids” can comprise proximal and more distal upstream elements. Promoter regulatory nucleic acids influence the transcription, RNA processing or stability, or translation of the associated coding sequence. Regulatory nucleic acids include enhancers, untranslated leader sequences, introns, exons, polyadenylation signal sequences and terminators. They include natural and synthetic sequences as well as sequences that can be a combination of synthetic and natural sequences. An “enhancer” is a nucleotide sequence that can stimulate promoter activity and can be an innate element of the promoter or a heterologous element inserted to enhance the level or tissue specificity of a promoter. The primary sequence can be present on either strand of a double-stranded DNA molecule, and is capable of functioning even when placed either upstream or downstream from the promoter. The meaning of the term “promoter” includes “transcription regulatory nucleic acids”, in particular transcription regulatory nucleic acids that involved RNA polymerase II.

A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. “Constitutive plant promoter” refers to a promoter that is able to express the open reading frame (ORF) that it controls in all or nearly all of the plant tissues during all or nearly all developmental stages of the plant (referred to as “constitutive expression”). “Regulated plant promoter” refers to promoters that direct gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. Some promoters preferentially initiate transcription in certain tissues, such as leaves, roots, seeds, fibers, xylem vessels, tracheids or sclerenchyma. Such promoters are referred to as “tissue preferred”. A “cell type” promoter primarily drives expression in certain cell types in one or more organs, for example, vascular cells in roots or leaves. An “inducible” plant promoter is a promoter, which is under environmental control. Examples of environmental conditions that may affect transcription by inducible promoters include anaerobic conditions, drought stress, abiotic stress, biotic stress or the presence of light. Promoters “regulated by light” include promoters that have increased transcription in the presence of light. Promoters regulated by light may include, but are not limited to, promoters regulating transcription of genes coding for proteins involved in photosynthesis such as the genes involved in photosystem I, photosystem II and the Calvin cycle. In general, promoters regulated by light drive high levels of transcription in green tissue such as leaf, stem, or seedling and low levels of transcription in other tissues such as, root, seed or embryo. Another type of promoter is a developmentally regulated promoter, for example, a promoter that drives expression during pollen development.

“Regulatory sequences” or “regulatory nucleic acids” refer to nucleotide sequences that contribute to the activity of a given gene as it relates to mRNA production, stability and translatability. Regulatory sequences include enhancers, promoters, translational enhancer sequences, introns, terminators and polyadenylation signal sequences. They include natural and synthetic sequences as well as sequences which may be a combination of synthetic and natural sequences. When a regulatory sequence is a combination of regulatory sequence elements, such as, a promoter, intron and terminator, the regulatory sequence elements are isolated or nonnaturally occurring from the same gene or different genes. For example, a promoter, intron and terminator sequence from the OsLHCA3 gene is isolated from the same the OsLHCA3 gene. Alternatively, the promoter could be from the OsLHCA3 gene, the intron from the OsLHCA4 gene and the terminator from the OsPSID gene. Light regulatory nucleic acids are regulatory elements that are preferentially transcribed in response to light and are therefore light inducible.

“Intron” refers to an intervening section of transcribed DNA that occurs almost exclusively within a eukaryotic gene, but which is not translated to amino acid sequences in the gene product. The introns are removed from the pre-mature mRNA through a process called splicing, which joins the exons to form an mRNA. For purposes of the presently disclosed subject matter, the definition of the term “intron” includes modifications to the nucleotide sequence of an intron derived from a target gene.

“Exon” refers to a section of transcribed DNA that is maintained in mRNA. Exons generally carry the coding sequence for a protein or part of the coding sequence. Exons are separated by intervening, non-coding sequences (introns). For purposes of the presently disclosed subject matter, the definition of the term “exon” includes modifications to the nucleotide sequence of an exon derived from a target gene.

A “terminator” refers to a nucleic acid capable of stopping gene transcription by RNA polymerase. Terminators typically consist of the 3′-UTR of a gene or coding sequence and about 1 kb of downstream sequence. For a review on terminators, please see, Richard and Manley (2009) Genes & Dev. 23:1247-1269.

As used herein, gene or trait “stacking” is combining desired genes or traits into one transgenic plant line. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. Another way to stack genes is by re-transforming a transgenic plant with another gene of interest. For example, gene stacking can be used to combine two different insect resistance traits, an insect resistance trait and a disease resistance trait, or a herbicide resistance trait. The use of a selectable marker in addition to a gene of interest would also be considered gene stacking.

Substantially identical: the phrase “substantially identical,” in the context of two nucleic acid or protein sequences, refers to two or more sequences or subsequences that have at least 60%, 80%, 90%, 95%, and 99% nucleotide or amino acid residue identity, when compared and aligned for maximum correspondence, as measured using a sequence comparison algorithms or by visual inspection. The substantial identity may exist over a region of the sequence that is at least about 50, 100, 150, 200, 250, 300, 350, 400, 450, 500, 550, 600, 650, 700, 750, 800, 850, 900, 950, 1000 residues in length. The sequences may be substantially identical over the entire length of the coding regions. Furthermore, substantially identical nucleic acid or protein sequences perform substantially the same function.

For purposes of the present invention, comparison of nucleotide sequences for determination of percent sequence identity to the promoter sequences disclosed herein can be made using the BLASTN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

Another indication that two nucleic acid sequences are substantially identical is that the two molecules hybridize to each other under stringent hybridization conditions. The phrase “hybridizing specifically to” refers to the binding, duplexing, or hybridizing of a molecule only to a particular nucleotide sequence under stringent hybridization conditions when that sequence is present in a complex mixture (e.g., total cellular) of DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2, “Overview of principles of hybridization and the strategy of nucleic acid probe assays”, Elsevier, New York. Generally, high stringency hybridization and wash conditions are selected to be about 5° C. lower than the thermal melting point (Tₘ) for the specific sequence at a defined ionic strength and pH. Typically, under high stringency conditions a probe will hybridize to its target subsequence, but to no other sequences.

The Tₘ is the temperature (under defined ionic strength and pH) at which 50% of the target sequence hybridizes to a perfectly matched probe. Very high stringency conditions are selected to be equal to the Tₘ for a particular probe. An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.1 M NaCl at 72° C. for about 15 minutes. An example of high stringency wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), high stringency conditions typically involve salt concentrations of less than about 1.0 M Na ion, typically about 0.01 to 1.0 M Na ion concentration (or other salts) at pH 7.0 to 8.3, and the temperature is typically at least about 30° C. High stringency conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under high stringency conditions are still substantially identical if the proteins that they encode are substantially identical. This occurs, for example, when a copy of a nucleic acid is created using the maximum codon degeneracy permitted by the genetic code.

Low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium. citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 42° C., and a wash in 0.1×SSC at 60 to 65° C.

The following are examples of sets of hybridization/wash conditions that may be used toidentify homologous nucleotide sequences that are substantially identical to reference nucleotide sequences of the present invention: a reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C.; 7% sodium dodecyl sulfate (SDS), 0.5 M NaPO₄, 1 mM EDTA at 50° C. with washing in 1×SSC, 0.1% SDS at 50° C.; 7% sodium dodecyl sulfate (SDS), 0.5 M NaPO₄, 1 mM EDTA at 50° C. with washing in 0.5×SSC, 0.1% SDS at 50° C.; 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

Specificity is typically the function of post-hybridization washes, the critical factors being the ionic strength and temperature of the final wash solution. For DNA-DNA hybrids, the Tₘ can be approximated from the equation of Meinkoth and Wahl Anal. Biochem. 138:267-284 (1984); TM 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. T is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, high stringency conditions are selected to be about 19° C. lower than the thermal melting point (Tₘ) for the specific sequence and its complement at a defined ionic strength and pH. However, very high stringency conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part 1, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2^{nd} ed., Cold Spring Harbor Laboratory Press, Plainview, N.Y.).

The “terminus” includes the 3′-untranslated sequence and the 3′ non-transcribed sequence, which extends 0.5 to 1.5 kb downstream of the transcription termination site. The terminus may include 3′ regulatory sequence.

A “synthetic or nonnaturally occurring gene cassette” will comprise in the 5′-3′ direction of transcription, a transcriptional and translational initiation region, a DNA sequence of interest, and a transcriptional and translational termination region functional in plants. The termination region may be native or physically or genetically linked with the transcriptional initiation region, may be native with the DNA sequence of interest, or may be derived from another source.

The “transcription initiation site” is the position surrounding the first nucleotide that is part of the transcribed sequence, which is also defined as position+1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

The term “transformation” refers to the transfer of a nucleic acid fragment into the genome of a host cell, resulting in genetically stable inheritance. “Transiently transformed” refers to cells in which transgenes and foreign DNA have been introduced (for example, by such methods as Agrobacterium-mediated transformation or biolistic bombardment), but not selected for stable maintenance. “Stably transformed” refers to cells that have been selected and regenerated on a selection media following transformation.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The term “translational enhancer sequence” refers to that DNA sequence portion of a gene between the promoter and coding sequence that is transcribed into RNA and is present in the fully processed mRNA upstream (5′) of the translation start codon. The translational enhancer sequence may affect processing of the primary transcript to mRNA, mRNA stability or translation efficiency.

As used herein, the term “recombinant” refers to a form of nucleic acid (e.g. DNA or RNA) and/or protein and/or an organism that would not normally be found in nature and as such was created by human intervention. Such human intervention may produce a recombinant nucleic acid molecule and/or a recombinant plant. As used herein, a “recombinant DNA molecule” is a DNA molecule comprising a combination of DNA molecules that would not naturally occur together and is the result of human intervention, e.g., a DNA molecule that is comprised of a combination of at least two DNA molecules heterologous to each other, and/or a DNA molecule that is artificially synthesized and comprises a polynucleotide that deviates from the polynucleotide that would normally exist in nature, and/or a DNA molecule that comprises a transgene artificially incorporated into a host cell's genomic DNA and the associated flanking DNA of the host cell's genome. An example of a recombinant DNA molecule is a DNA molecule resulting from the insertion of the transgene into a plant's genomic DNA, which may ultimately result in the expression of a recombinant RNA and/or protein molecule in that organism. As used herein, a “recombinant plant” is a plant that would not normally exist in nature, is the result of human intervention, and contains a transgene and/or heterologous DNA molecule incorporated into its genome. As a result of such genomic alteration, the recombinant plant is distinctly different from the related wildtype plant.

“Vector” is defined to include, inter alia, any plasmid, cosmid, phage or Agrobacterium binary vector in double or single stranded linear or circular form which may or may not be self-transmissible or mobilizable, and which can transform a prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which may be selected from actinomycetes and related species, bacteria and eukaryotic species (e.g. plant, mammalian, yeast or fungal cells).

The term “plant” refers to any plant, particularly to agronomically useful plants (e.g. seed plants), and “plant cell” is a structural and physiological unit of the plant, which comprises a cell wall but may also refer to a protoplast. The plant cell may be in the form of an isolated single cell or a cultured cell, or as a part of higher organized units such as for example, a plant tissue, or a plant organ differentiated into a structure that is present at any stage of a plant's development. The promoters and compositions described herein may be utilized in any plant. Examples of plants that may be utilized in contained embodiments herein include, but are not limited to, maize (corn), wheat, rice, barley, soybean, cotton, sorghum, beans in general, rape/canola, alfalfa, flax, sunflower, safflower, millet, rye, sugarcane, sugar beet, cocoa, tea, tropical sugar beet, Brassica spp., cotton, coffee, sweet potato, flax, peanut, clover; vegetables such as lettuce, tomato, cucurbits, cassava, potato, carrot, radish, pea, lentils, cabbage, cauliflower, broccoli, Brussel sprouts, peppers, and pineapple; tree fruits such as citrus, apples, pears, peaches, apricots, walnuts, avocado, banana, and coconut; and flowers such as orchids, carnations and roses. Other plants useful in the practice of the invention include perennial grasses, such as switchgrass, prairie grasses, Indiangrass, Big bluestem grass, miscanthus and the like. It is recognized that mixtures of plants can be used.

As used herein, “plant tissue”, “plant cell”, “plant material,” “plant part” or “plant portion thereof” means plant cells, plant protoplasts, plant cell tissue cultures, differentiated and undifferentiated tissues from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, tubers, rhizomes and the like.

A transcription regulating nucleic acid may comprise at least one promoter sequence localized upstream of the transcription start of the respective gene and is capable of inducing transcription of downstream sequences. The transcription regulating nucleic acid may comprise the promoter sequence of said genes but may further comprise other elements such as the 5′-untranslated sequence, enhancer sequences, intron, exon, and/or even comprise intron and exons of the associated genomic gene.

Promoters can comprise several regions that play a role in function of the promoter. Some of these regions are modular, in other words they can be used in isolation to confer promoter activity or they can be assembled with other elements to construct new promoters. The first of these promoter regions lies immediately upstream of the coding sequence and forms the “core promoter region” containing consensus sequences, normally the region immediately upstream of the coding sequence. The core promoter region typically contains an initiator element as well as the initiation site. The precise length of the core promoter region is not fixed. Such a region is normally present, with some variation, in most promoters. The core promoter region is often referred to as a minimal promoter region because it is functional on its own to promote a basal level of transcription.

The presence of the core promoter region defines a sequence as being a promoter: if the region is absent, the promoter is non-functional. The core region acts to attract the general transcription machinery to the promoter for transcription initiation. However, the core promoter region is typically not sufficient to provide promoter activity at a desired level or in a regulated manner. A series of regulatory sequences, often upstream of the core, constitute the remainder of the promoter. The regulatory sequences can determine expression level, the spatial and temporal pattern of expression and, for a subset of promoters, expression under inductive conditions (regulation by external factors such as light, temperature, chemicals and hormones). Regulatory sequences can be short regions of DNA sequence 6-100 base pairs that define the binding sites for trans-acting factors, such as transcription factors. Regulatory sequences can also be enhancers, longer regions of DNA sequence that can act from a distance from the core promoter region, sometimes over several kilobases from the core region. Regulatory sequence activity can be influenced by trans-acting factors including but not limited to general transcription machinery, transcription factors and chromatin assembly factors. Transcription factor binding “motifs” represent the differences in the sequence that a transcription factor binds in different promoters by using IUPAC codes to represent the degenerate positions such as “R” represents “A” or “G”.

As used herein, a “control plant” may be a non-transgenic plant of the parental line used to generate a transgenic plant herein. A control plant may in some cases be a transgenic plant line that includes an empty vector or marker gene, but does not contain the recombinant polynucleotide of the present invention that is expressed in the transgenic plant being evaluated. In general, a control plant is a plant of the same line or variety as the transgenic plant being tested, lacking the specific trait-conferring, recombinant DNA that characterizes the transgenic plant. Such a progenitor plant that lacks that specific trait-conferring recombinant DNA can be a natural, wild-type plant, an elite, non-transgenic plant, or a transgenic plant without the specific trait-conferring, recombinant DNA that characterizes the transgenic plant. The progenitor plant lacking the specific, trait-conferring recombinant DNA can be a sibling of a transgenic plant having the specific, trait-conferring recombinant DNA. Such a progenitor sibling plant may include other recombinant DNA.

The term “modulate” (and grammatical variations) refers to an increase or decrease. As used herein, the terms “increase,” “increases,” “increased,” “increasing” and similar terms indicate an elevation of at least about 5%, 10%, 15%, 20%, 25%, 30%, 35%, 40%, 45%, 50%, 55%, 60%, 75%, 80%, 85%, 90%, 95%, 100%, 150%, 200%, 300%, 400%, 500% or more as compared to a control (e.g., a plant that does not comprise at least one nonnaturally occurring nucleic acid of the present invention).

As used herein, the terms “reduce,” “reduces,” “reduced,” “reduction” and similar terms mean a decrease of at least about 5%, 10%, 15%, 20%, 25%, 30%, 35%, 40%, 45%, 50%, 55%, 60%, 75%, 80%, 85%, 90%, 95%, 100%, 150%, 200%, 300%, 400%, 500% or more as compared to a control (e.g., a plant that does not comprise at least one nonnaturally occurring nucleic acid of the present invention). In particular embodiments, the reduction results in no or essentially no (i.e., an insignificant amount, e.g., less than about 10%, less than about 5% or even less than about 1%) detectable activity or amount.

As used herein the phrase “plant biomass” refers to the amount (measured in grams of air-dried or Heat-dried tissue) of a tissue produced from the plant in a growing season, which could also determine or affect the plant yield or the yield per growing area.

As used herein, “yield” may include reference to bushels per acre of a grain crop at harvest, as adjusted for grain moisture (15.5% typically for maize, for example), and the volume of biomass generated (for forage crops such as alfalfa and plant root size for multiple crops). Grain moisture is measured in the grain at harvest. The adjusted test weight of grain is determined to be the weight in pounds per bushel, adjusted for grain moisture level at harvest. Biomass is measured as the weight of harvestable plant material generated. Yield can be affected by many properties including without limitation, plant height, pod number, pod position on the plant, number of internodes, incidence of pod shatter, grain size, efficiency of nodulation and nitrogen fixation, efficiency of nutrient assimilation, carbon assimilation, plant architecture, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill. Yield of a plant of the can be measured in a number of ways, including test weight, seed number per plant, seed weight, seed number per unit area (i.e. seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Moreover a bushel of corn is defined by law in the State of Iowa as 56 pounds by weight, a useful conversion factor for corn yield is: 100 bushels per acre is equivalent to 6.272 metric tons per hectare. Other measurements for yield are common practice in the art. In certain embodiments of the invention yield may be increased in stressed and/or non-stressed conditions.

Highly active light regulated, green tissue preferred expression cassettes are desirable tools for bioengineering plants for a number of traits, for example, improved yield or drought tolerance. Genes expressed in these cassettes could contribute to photosynthesis or cause the plant to make better use of the energy produced by photosynthesis. Light regulated promoters might be found driving the expression of native genes for photosystem I, photosystem II, or Calvin Cycle proteins.

One approach to designing plant expression cassettes is to try to get all of the regulatory sequence guiding the expression of a gene by including 5′ flanking sequence, 3′ flanking sequence and intron sequence. Genome analysis shows that genes are dispersed at 4-6 kb intervals in rice (Delseny, Current Opinion Plant Biology 6 (2): 101-105 (2003)). A plant gene can be broken into three basic components: the promoter, the coding sequence and the terminator. The promoter may consist of 5′-upstream regulatory (non-transcribed) sequence, generally 1.0-2.5 kb, and the 5′-UTR. The coding sequence consists of the exons and introns between the translation start and stop codons. The terminator consists of the 3′-UTR and about 1 kb of downstream sequence. These components contain virtually all of the necessary gene regulatory information and can be used to design transgene expression cassettes that replicate or recapitulate the expression profile of a gene from which the transgene regulatory sequence was derived. This model has been applied in both dicots (U.S. Pat. No. 6,100,450) and monocots (U.S. Pat. No. 8,129,588).

Each cassette is based on a unique plant gene derived from rice, maize, or sugar cane. Construct design is modeled on plant gene structure, described above. Where possible, attention was paid to transcribed sequence to reduce the occurrence of sequence repeats of more than 15 nucleotides. Modifications were achieved by substituting adenosine for thymidine or cytidine for guanidine (and vice versa) at 15 base intervals, except in introns, to minimize gene silencing (Carrington et al., Science 301 (5631): 336-338 (2003)). Also sequence surrounding the intended translation start codon can be optimized following the guidelines of Kozak (Kozak, Gene 299(1-2): 1-34(2002)). This design strategy eliminates repetitive sequence that could trigger gene silencing and produces a construct that looks more like plant genomic DNA and less like plant pathogen DNA. The constructs are assembled in a binary vector and transformed into maize using standard agrobacterium procedures.

Expression cassettes can be introduced into the plant cell in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptll gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens. These typically carry at least one T-DNA border sequence and include vectors such as pBIN19 (Bevan, Nucl. Acids Res. (1984)). For the construction of vectors useful in Agrobacterium transformation, see, for example, US Patent Application Publication No. 2006/0260011, herein incorporated by reference.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. For the construction of such vectors, see, for example, US Application No. 20060260011, herein incorporated by reference.

Transformation techniques for plants are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

The plants obtained via transformation with a nucleic acid sequence of the present invention can be any of a wide variety of plant species; however, the plants used in the method of the invention can be selected from the list of agronomically important target crops set forth supra. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, N Y (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

It is specifically contemplated that one could mutagenize a promoter to potentially improve the utility of the elements for the expression of transgenes in plants. The mutagenesis of these elements can be carried out at random and the mutagenized promoter sequences screened for activity in a trial-by-error procedure. Alternatively, particular sequences which provide the promoter with desirable expression characteristics, or the promoter with expression enhancement activity, could be identified and these or similar sequences introduced into the promoter via mutation. It is further contemplated that one could mutagenize these sequences in order to enhance their expression of transgenes in a particular species. The means for mutagenizing a DNA segment encoding a promoter sequence of the current invention are well-known to those of skill in the art. As indicated, modifications to promoter or other regulatory element may be made by random, or site-specific mutagenesis procedures. The promoter and other regulatory element may be modified by altering their structure through the addition or deletion of one or more nucleotides from the sequence which encodes the corresponding unmodified sequences.

Mutagenesis may be performed in accordance with any of the techniques known in the art, such as, and not limited to, synthesizing an oligonucleotide having one or more mutations within the sequence of a particular regulatory sequence. In particular, site-specific mutagenesis is a technique useful in the preparation of promoter mutants, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

Where a clone comprising a promoter has been isolated in accordance with the instant invention, one may wish to delimit the essential promoter regions within the clone. One efficient, targeted means for preparing mutagenized promoters relies upon the identification of putative regulatory elements within the promoter sequence. This can be initiated by comparison with promoter sequences known to be expressed in similar tissue specific or developmentally unique patterns. Sequences which are shared among promoters with similar expression patterns are likely candidates for the binding of transcription factors and are thus likely elements which confer expression patterns. Confirmation of these putative regulatory elements can be achieved by deletion analysis of each putative regulatory sequence followed by functional analysis of each deletion construct by assay of a reporter gene which is functionally attached to each construct. As such, once a starting promoter sequence is provided, any of a number of different deletion mutants of the starting promoter could be readily prepared.

Functional equivalent fragments of the OsLHCA3, OsLHCA4, OsPSAK or OsPSID promoters may be 50, 100, 150, 200, 250, 300, 350, 400, 450, 500, 550, 600, 650, 700, 750, 800, 850, 900, 950, 1000 or more base pairs. Narrowing the transcription regulating nucleic acid to its essential, transcription mediating elements can be realized in vitro by trial-and-error deletion mutations, or in silico using promoter element search routines. Regions essential for promoter activity often demonstrate clusters of certain, known promoter elements. Such analysis can be performed using available computer algorithms such as PLACE (“Plant Cis-acting Regulatory DNA Elements”; Higo Nucl. Acids Res. 27 (1): 297-300 (1999), the BIOBASE database “Transfac” Wingender Nucl. Acids Res. 29 (1): 281-283 (2001) or the database PlantCARE Lescot Nucl. Acids Res. 30 (1): 325-327 (2002).

For example, functional borders, genetic fine structure, and distance requirements of cis elements mediating light responsiveness of the parsley chalcone synthase promoter Proc Natl Acad Sci USA 87:5387-5391(1990); Terzaghi W B, Cashmore A R Light-regulated transcription Annu Rev Plant Physiol Plant Mol Biol 46:445-474 (1995); Nakashima K, Fujita Y, Katsura K, Maruyama K, Narusaka Y, Seki M, Shinozaki K, Yamaguchi-Shinozaki K. Transcriptional regulation of ABI3- and ABA-responsive genes including RD29B and RD29A in seeds, germinating embryos, and seedlings of Arabidopsis. Plant Mol Biol. 60: 51-68 (2006); Piechulla B, Merforth N, Rudolph B Identification of tomato Lhc promoter regions necessary for circadian expression Plant Mol Biol 38:655-662 (1998); Villain P, Mache R, Zhou DX The mechanism of GT element-mediated cell type-specific transcriptional control J Biol Chem 271:32593-32598 (1996); Le Gourrierec J, Li Y F, Zhou DX Transcriptional activation by Arabidopsis GT-1 may be through interaction with TFIIA-TBP-TATA complex Plant J 18:663-668 (1999); Buchel A S, Brederode F T, Bol J F, Linthorst HJM Mutation of GT-1 binding sites in the Pr-1A promoter influences the level of inducible gene expression in vivo Plant Mol Biol 40:387-396 (1999); Zhou DX Regulatory mechanism of plant gene transcription by GT-elements and GT-factors Trends in Plant Science 4:210-214 (1999); Giuliano G, Pichersky E, Malik V S, Timko M P, Scolnik P A, Cashmore AR An evolutionarily conserved protein binding sequence upstream of a plant light regulated gene. Proc Natl Acad Sci USA 85:7089-7093 (1988); Donald RGK, Cashmore AR Mutation of either G box or 1 box sequences profoundly affects expression from the Arabidopsis rbcS-1A promoter. EMBO J 9:1717-1726 (1990); Rose A, Meier I, Wienand U The tomato I-box binding factor LeMYBI is a member of a novel class of Myb-like proteins Plant J 20: 641-652 (1999); Martinez-Hernandez A, Lopez-Ochoa L, Arguello-Astorga, G,Herrera-Estrella L. Functional properties and regulatory complexity of a minimal RBCS light-responsive unit activated by phytochrome, cryptochrome, and plastid signals. Plant Physiol. 128:1223-1233 (2002); Nakamura M, Tsunoda T, Obokata J Photosynthesis nuclear genes generally lack TATA-boxes: a tobacco photosystem I gene responds to light through an initiator Plant J 29: 1-10 (2002); Castresana C, Garcia-Luque I, Alonso E, Malik V S, Cashmore A R Both positive and negative regulatory elements mediate expression of a photoregulated CAB gene from Nicotiana plumbaginifolia EMBO J 7:1929-1936 (1988); Hudson M E, Quail P H. Identification of promoter motifs involved in the network of phytochrome A-regulated gene expression by combined analysis of genomic sequence and microarray data. Plant Physiol. 133: 1605-1616 (2003); Jiao Y, Ma L, Strickland E, Deng X W. Conservation and Divergence of Light-Regulated Genome Expression Patterns during Seedling Development in Rice and Arabidopsis. Plant Cell. 17: 3239-3256 (2005)).

Promoter activity can be routinely confirmed by expression assays, for example, as described in the Examples section herewith. In addition, modification of promoter sequences without loss of activity is routine in the art. For example, the well-known CaMV 35S promoter has been shown to retain promoter activity when fragmented into two domains, with Domain A (−90 to +8) able to confer expression primarily in root tissues (Benfey et. al., (1989) EMBO J 8(8):2195-2202 and Domain B (−343 to −90) conferring expression in most cell types of leaf, stem and root vascular tissues. A CaMV promoter has been truncated to a-46 promoter and still retains, although reduced, correct promoter activity (Odell et. al., (1985) Nature 313:810-812).

Welsch et. al. describe the creation of multiple deletion fragments of an Arabidopsis thaliana phytoene synthase gene promoter (Welsch et. al. (2003) Planta 216:523-534). Using truncation studies, Welsch et. al. showed that as little as 11% of the promoter needed to be retained in order to observe some promoter activity. The deletion analysis of promoters from the cab 1A, cab 1B, cab8 and cab 11 genes from the tomato light harvesting complex of genes determined which deletion would affect circadian expression (Piechulla, et. al. (1998) Plant Molecular Biology 38:655-662). A deletion of approximately 775 bp could be made from a 1058 bp plant promoter designated AtEXP18 without significantly reducing promoter activity (Cho and Cosgrove (2002) Plant Cell 14:3237-3253). In addition, the authors showed that numerous substitution mutations could be made in a fragment of AtEXP18, while retaining full promoter activity and in some cases increasing activity.

The invention disclosed herein provides polynucleotide molecules comprising regulatory element fragments that may be used in constructing novel chimeric regulatory elements. Novel combinations comprising fragments of these polynucleotide molecules and at least one other regulatory element or fragment can be constructed and tested in plants and are considered to be within the scope of this invention. Thus the design, construction, and use of chimeric regulatory elements is one embodiment of this invention. Promoters of the present invention include homologues of cis elements known to effect gene regulation that show homology with the promoter sequences of the present invention. These cis elements include but are not limited to light regulatory elements.

Functional equivalent fragments of one of the transcription regulating nucleic acids described herein comprise at least 50, 100, 150, 200, 250, 300, 350, 400, 450, 500, 550, 600, 650, 700, 750, 800, 850, 900, 950, or 1000 base pairs of a transcription regulating nucleic acid as described by SEQ ID NOS. 1 to 15. Equivalent fragments of transcription regulating nucleic acids, which are obtained by deleting the region encoding the 5′-untranslated region of the mRNA, would then only provide the (untranscribed) promoter region. The 5′-untranslated region can be easily determined by methods known in the art (such as 5′-RACE analysis). Accordingly, some of the transcription regulating nucleic acids, described herein, are equivalent fragments of other sequences.

As indicated above, deletion mutants of the promoter of the invention also could be randomly prepared and then assayed. Following this strategy, a series of constructs are prepared, each containing a different portion of the promoter (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes.

An expression cassette as described herein may comprise further regulatory elements. The term in this context is to be understood in the broad meaning comprising all sequences which may influence construction or function of the expression cassette. Regulatory elements may, for example, modify transcription and/or translation in prokaryotic or eukaryotic organisms. The expression cassette described herein may be downstream (in 3′-direction) of the nucleic acid sequence to be expressed and optionally contain additional regulatory elements, such as transcriptional or translational enhancers. Each additional regulatory element may be operably liked to the nucleic acid sequence to be expressed (or the transcription regulating nucleotide sequence). Additional regulatory elements may comprise additional promoters, minimal promoters, promoter elements, or transposon elements which may modify or enhance the expression regulating properties. The expression cassette may also contain one or more introns, one or more exons and one or more terminators.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters. Promoters which are useful for plant transgene expression include those that are inducible, viral, synthetic, constitutive (Odell Nature 313: 810-812 (1985)), temporally regulated, spatially regulated, tissue specific, and spatial temporally regulated. Using the regulatory elements described herein, numerous agronomic genes can be expressed in transformed plants. More particularly, plants can be genetically engineered to express various phenotypes of agronomic interest. Exemplary genes implicated in this regard include, but are not limited to, those categorized below.

### 1. Pests or Disease Resistance Nucleic Acids, for Example:

(A) Plant disease resistance nucleic acids. Plant defenses are often activated by specific interaction between the product of a disease resistance gene (R) in the plant and the product of a corresponding avirulence (Avr) gene in the pathogen. A plant can be transformed with a cloned resistance gene to engineer plants that are resistant to specific pathogen strains. See, for example Jones et al., Science 266: 789 (1994) (cloning of the tomato Cf-9 gene for resistance to Cladosporium fulvum); Martin et al., Science 262: 1432 (1993) (tomato Pto gene for resistance to Pseudomonas syringae pv. tomato encodes a protein kinase); Mindrinos et al., Cell 78: 1089 (1994) (Arabidopsis RSP2 gene for resistance to Pseudomonas syringae). A developmental-arrestive protein produced in nature by a pathogen or a parasite. Thus, fungal endo.alpha.-1,4-D-polygalacturonases facilitate fungal colonization and plant nutrient release by solubilizing plant cell wall homo-.alpha.-1,4-D-galacturonase. See Lamb et al., Bio/Technology 10: 1436 (1992). The cloning and characterization of a gene which encodes a bean endopolygalacturonase-inhibiting protein is described by Toubart et al., Plant J. 2: 367 (1992). A molecule that stimulates signal transduction. For example, see the disclosure by Botella et al., Plant Molec. Biol. 24: 757 (1994), of nucleotide sequences for mung bean calmodulin cDNA clones, and Griess et al., Plant Physiol. 104: 1467 (1994), who provide the nucleotide sequence of a maize calmodulin cDNA clone. A hydrophobic moment peptide. See PCT application WO95/16776 (disclosure of peptide derivatives of Tachyplesin which inhibit fungal plant pathogens) and PCT application WO95/18855 (teaches synthetic antimicrobial peptides that confer disease resistance), the respective contents of which are hereby incorporated by reference. A membrane permease, a channel former or a channel blocker. For example, see the disclosure by Jaynes et al., Plant Sci. 89: 43 (1993), of heterologous expression of a cecropin-.beta. lytic peptide analog to render transgenic tobacco plants resistant to Pseudomonas solanacearum. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28: 451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland, 1994) (enzymatic inactivation in transgenic tobacco via production of single-chain antibody fragments). A virus-specific antibody. See, for example, Tavladoraki et al., Nature 366: 469 (1993), who show that transgenic plants expressing recombinant antibody genes are protected from virus attack. A developmental-arrestive protein produced in nature by a plant. For example, Logemann et al., Bio/Technology 10: 305 (1992), have shown that transgenic plants expressing the barley ribosome-inactivating gene have an increased resistance to fungal disease.

(B) Pest Resistance Nucleic Acids. A Bacillus thuringiensis protein, a derivative thereof or a synthetic polypeptide modeled thereon. See, for example, Geiser et al., Gene 48: 109 (1986), who disclose the cloning and nucleotide sequence of a Bt.delta.-endotoxin gene. Moreover, DNA molecules encoding .delta.-endotoxin genes can be purchased from American Type Culture Collection (Rockville, Md.), for example, under ATCC Accession Nos. 40098, 67136, 31995 and 31998. A lectin. See, for example, the disclosure by Van Damme et al., Plant Molec. Biol. 24: 25 (1994), who disclose the nucleotide sequences of several Clivia miniata mannose-binding lectin genes. A vitamin-binding protein, such as avidin. See PCT application US93/06487 the contents of which are hereby incorporated by. The application teaches the use of avidin and avidin homologues as larvicides against insect pests. An enzyme inhibitor, for example, a protease inhibitor or an amylase inhibitor. See, for example, Abe et al., J. Biol. Chem. 262: 16793 (1987) (nucleotide sequence of rice cysteine proteinase inhibitor), Huub et al., Plant Molec. Biol. 21: 985 (1993) (nucleotide sequence of cDNA encoding tobacco proteinase inhibitor I), and Sumitani et al., Biosci. Biotech. Biochem. 57: 1243 (1993) (nucleotide sequence of Streptomyces nitrosporeus.alpha.-amylase inhibitor). An insect-specific hormone or pheromone such as an ecdysteroid and juvenile hormone, a variant thereof, a mimetic based thereon, or an antagonist or agonist thereof. See, for example, the disclosure by Hammock et al., Nature 344: 458 (1990), of baculovirus expression of cloned juvenile hormone esterase, an inactivator of juvenile hormone. An insect-specific peptide or neuropeptide which, upon expression, disrupts the physiology of the affected pest. For example, see the disclosures of Regan, J. Biol. Chem. 269: 9 (1994) (expression cloning yields DNA coding for insect diuretic hormone receptor), and Pratt et al., Biochem. Biophys. Res. Comm. 163: 1243 (1989) (an allostatin is identified in Diploptera puntata). See also U.S. Pat. No. 5,266,317 to Tomalski et al., who disclose genes encoding insect-specific, paralytic neurotoxins. Insect-specific venom produced in nature by a snake, a wasp, etc. For example, see Pang et al., Gene 116: 165 (1992), for disclosure of heterologous expression in plants of a gene coding for a scorpion insect toxic peptide. An enzyme responsible for a hyperaccumulation of a monterpene, a sesquiterpene, a steroid, hydroxamic acid, a phenylpropanoid derivative or another non-protein molecule with insecticidal activity. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23: 691 (1993), who teach the nucleotide sequence of a cDNA encoding tobacco hookworm chitinase, and Kawalleck et al., Plant Mole. Biol. 21: 673 (1993), who provide the nucleotide sequence of the parsley ubi4-2 polyubiquitin gene.

### 2. Herbicide Resistance Nucleic Acids, for Example:

An herbicide that inhibits the growing point or meristem, such as an imidazalinone or a sulfonylurea. Exemplary genes in this category code for mutant ALS and AHAS enzyme as described, for example, by Lee et al., EMBO J. 7: 1241 (1988), and Miki et al., Theor Appl. Genet. 80: 449 (1990), respectively. Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cycloshexones (ACCase inhibitor-encoding genes). See, for example, U.S. Pat. No. 4,940,835 to Shah et al., which discloses the nucleotide sequence of a form of EPSP which can confer glyphosate resistance. A DNA molecule encoding a mutant aroA gene can be obtained under ATCC accession No. 39256, and the nucleotide sequence of the mutant gene is disclosed in U.S. Pat. No. 4,769,061 to Comai. European patent application No. 0 333 033 and U.S. Pat. No. 4,975,374 describe nucleotide sequences of glutamine synthetase genes which confer resistance to herbicides such as L-phosphinothricin. The nucleotide sequence of a phosphinothricin-acetyl-transferase gene is provided in European application No. 0 242 246; De Greef et al., Bio/Technology 7: 61 (1989), describe the production of transgenic plants that express chimeric bar genes coding for phosphinothricin acetyl transferase activity. Exemplary of genes conferring resistance to phenoxy proprionic acids and cycloshexones, such as sethoxydim and haloxyfop, are the Accl-S1, Accl-S2 and Accl-S3 genes described by Marshall et al., Theor. Appl. Genet. 83: 435 (1992). An herbicide that inhibits photosynthesis, such as a triazine (psbA and gs+ genes) and a benzonitrile (nitrilase gene). Przibilla et al., Plant Cell 3: 169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285: 173 (1992).

### 3. Value-Added Trait Nucleic Acids, for Example:

Modified fatty acid metabolism, for example, by transforming a plant with an antisense gene of stearoyl-ACP desaturase to increase stearic acid content of the plant. See Knultzon et al., Proc. Natl. Acad. Sci. USA 89: 2624 (1992). Introduction of a phytase-encoding gene would enhance breakdown of phytate, adding more free phosphate to the transformed plant. For example, see Van Hartingsveldt et al., Gene 127: 87 (1993), for a disclosure of the nucleotide sequence of an Aspergillus niger phytase gene. Modified carbohydrate composition effected, for example, by transforming plants with a gene coding for an enzyme that alters the branching pattern of starch. See Shiroza et al., J. Bacteriol. 170: 810 (1988) (nucleotide sequence of Streptococcus mutans fructosyltransferase gene), Steinmetz et al., Mol. Gen. Genet. 200: 220 (1985) (nucleotide sequence of Bacillus subtilis levansucrase gene), Pen et al., Bio/Technology 10: 292 (1992) (production of transgenic plants that express Bacillus licheniformis.alpha.-amylase), Elliot et al., Plant Molec. Biol. 21: 515 (1993) (nucleotide sequences of tomato invertase genes) and Fisher et al., Plant Physiol. 102: 1045 (1993) (maize endosperm starch branching enzyme II).

### 4. Photoassimilation Regulation Nucleic Acids, for Example:

Any of the enzymes or genes involved in the C₃, C₄ or CAM photosynthesis/photorespiration pathway may be operably linked to any of the regulatory nucleic acids described herein. Enzymes may include rubisco (ribulose bisphosphate carboxylase/oxygenase, EC 4.1.1.39), phosphoglycollate phosphatase (EC 3.1.3.18), (S)-2-hydroxy-acid oxidase (EC 1.1.3.15), glycine transaminase (EC 2.6.1.4), serine-glyoxylate aminotransferase (EC 2.6.1.45), glycerate dehydrogenase (EC 1.1.1.29), glycerate kinase (2.7.1.31); phosphoenolpyruvate carboxylase (PEPC, EC 4.1.1.31), NADP-dependent malic enzyme (NADPMD) or malate dehydrogenase (EC 1.1.1.40, EC 1.1.1.82), phosphoglycerate kinase (PGK, EC 2.7.2.3), sedoheptulose-1,7-bisphosphatase (SBP, EC 3.1.3.37), fructose-1, 6-bisphosphate phosphatase (FBPase, EC 3.1.3.11), phosphoribulokinase (PRK, EC 2.7.1.19), pyruvate, orthophosphate dikinase (PPDK, EC 2.7.9.1), and the like. Numerous examples of the photoassimilation regulation genes can be found in the literature. The BRENDA database (brenda.enzymes.org) can be queried for sequence information on many of the genes involved in the photosynthesis/photorespiration pathways. In particular, examples of PRK, SBP, PGK and NADPME from maize can be found in WO2012061585, which is hereby incorporated by reference. Typical C₃ plants include wheat, rice, soybean and potato. Typical C₄ plants are primarily monocotyledonous plants include maize, sugarcane, sorghum, amaranth, other grasses and sedges. Typical CAM plants are pineapple, epiphytes, succulent xerophytes, hemiepiphytes, lithophytes, terrestrial bromeliads, wetland plants, Mesembryanthemum crystallinum, Dodoneaea viscosa, and Sesuvium portulacastrum. It is possible to express photoassimilation regulation genes from one type of plant in another. For example, C₄-cycle enzymes have been introduced into C₃ plants. For a review, please see Hausler, et. al. (2002) J of Experimental Botany, Vol. 53, No. 369, pp. 591-607).

### 5. Yield Increasing or Stress Tolerant Nucleic Acids

There are a number of nucleic acids that may provide improved yield, such as, improved grain yield or biomass. In addition, there are a number of nucleic acids that improve a plants ability to yield under a number of abiotic stresses, such as, drought, salinity, heat, reduced nitrogen, shade tolerance and the like. For example, U.S. Pat. Nos. 7,030,294; 6,686,516; 6,566,511, 5,925,804; 6,833,490; 7,247,770 and US Patent Publication No. 2010/0205692, describe the use of genes of the trehalose pathway for increasing yield and improving stress tolerance. U.S. Pat. Nos. 7,109,033; 7,692,065; 7,732,667 and US Patent Publication Nos. 2003/303589; 2003/299859 describe a number of plant genes for improving a plant's response to stress. Additional genes capable of conferring stress tolerance include, LNT1 gene for improving NUE (WO 2010/031312); GMWRKY54 gene (WO 2009/057061); genes for inhibiting ammonia (US Patent Publication No. 2011/0030099); OsGATA for nitrogen use efficiency (U.S. Pat. No. 7,554,018) and the like.

The foregoing examples described herein are for illustrative purposes only and are not intended to be limiting.

The following Examples provide illustrative embodiments. In light of the invention and the general level of skill in the art, those of skill will appreciate that the following Examples are intended to be exemplary only and that numerous changes, modifications, and alterations can be employed without departing from the scope of the presently claimed subject matter.

Unless indicated otherwise, The recombinant DNA steps carried out for the purposes of the present invention, such as, for example, restriction cleavages, agarose gel electrophoresis, purification of DNA fragments, linking DNA fragments, transformation of E. coli cells, growing bacteria, and sequence analysis of recombinant DNA, are carried out as described by Sambrook (1989).

### Example 1: Identification of Trait Protein Coding Sequences

Rice was selected as the donor organism for FBPase, SBPase and AGPase. The FBPase is GenBank Accession Q40677.2 (Tsutsumi et al., 1994). The SBPase selected is GenBank Accession Q84JG8. Two papers (Odhan et al., 2005; Lee et al., 2007) describe the rice AGPase gene family. According to these papers the leaf isoform, which localizes to plastids, consists of the large subunit gene AGPL3 (GenBank Accession BAG91362.1) and the small subunit gene AGPS2a (GenBank Accession AK071826.1). The coding sequence of these genes was optimized for efficient codon utilization in a dicot, such as, soybean. The optimized polynucleotide sequences are described in SEQ ID NOS: 1, 3, 5 and 7. The resulting polypeptide sequences are described in SEQ ID NOS: 2, 4, 6 and 8.

### Example 2: Identification of Trait Regulatory Sequences

It was determined by using the Zhu evolutionary algorithm, (please see, Zhu et al., ((2007) Plant Physiol 145: 513) that AGPase will need to be boosted approximately 4 fold higher than endogenous levels, SBPase will need to be boosted the approximately 4 fold higher than endogenous levels, and FBPase will need to be boosted approximately 2 fold higher than endogenous levels.

Promoters were selected based on linkage to photoassimilation and whether they provide the appropriate spatial and temporal regulation. Probe sequences listed in Table 1 were based well characterized proteins involved in plant photosynthesis. The amino acid sequences for Hordeum vulgare (barley) Photosystem I reaction center subunit psaD (PSID) with Swiss-Prot ID P36213.1, the Hordeum vulgare Photosystem I reaction center subunit psaK (PSAK) with Swiss-Prot ID P36886.1 (formerly Swiss-Prot ID A48527), the Pisum sativum (pea) light harvesting protein of photosystem I LHCA3(LHCA3) with Genbank ID AAA84545.1, and the Hordeum vulgare chlorophyll a/b-binding protein precursor LHCA4 (LHCA4) with Genbank ID AAF90200.1 were used in a tBLASTn search of rice gene databases to find the corresponding rice genes. cDNAs representing the nearest rice homologs are indicated in Table 1. gDNAs for each gene were identified, annotated and used to define their corresponding regulatory sequence. EST and gene expression profiling data in Table 2 confirm each gene is primarily active in green tissue.

The following promoters/terminators were identified and then isolated from rice. The OsLHCA3 promoter from a rice light harvesting protein of photosystem I gene (SEQ ID NO: 9), the OsLHCA4 promoter from a rice chlorophyll a/b-binding protein precursor (SEQ ID NO: 10); the OsPSAK promoter from a rice Photosystem I reaction center subunit gene (SEQ ID NO: 11) and the OsPSID promoter from a rice Photosystem I reaction center subunit gene (SEQ ID NO: 12).

TABLE 1
Identification of rice genes linked to photoassimilation

| | | Probe | | | | |
|---|---|---|---|---|---|---|
| | Probe | GenBank | Rice | Rice 24K | Rice 50K | Sequencher |
| Probe | Source | Accession | cDNA ID | Chip ID | Chip ID | File |
| PSI D ferredoxin | barley | P36213 | 104557 | OS000721_at | Os1013373_at | OspsaD |
| docking protein | | | | | | |
| psaK PSI protein | barley | A48527 | 100698 | OS001237.1_at | Os1016452_at | OspsaK |
| LHCA3 protein | pea | AAA84545 | 106085 | OS000810_at | Os1010826_at | OsLHCA3 |
| LHCA4 protein | barley | AAF90200 | 103081 | OS000854.1_at | Os1011693_at | OsLHCA4 |

TABLE 2
Characterization of rice genes linked to photoassimilation

| | EST ANALYSIS | | | | EXPRESSION PROFILING^{b} | | | | | |
|---|---|---|---|---|---|---|---|---|---|---|
| | green tissue^{a} | | | EB/IM seed | 26K Chip | | | 50K Chip | | |
| Rice Gene | Total ESTs | (%) | root | (%) | leaf | root | EB | leaf | root | EB |
| PSI D | 31 | 87.1 | 3.2 | 0.0 | 52.9 | 1.9 | 1.7 | 53.4 | 1.3 | 1.0 |
| ferredoxin | | | | | | | | | | |
| docking | | | | | | | | | | |
| protein | | | | | | | | | | |
| psaK PSI | 100 | 81.0 | 0.0 | 4.0 | 37.7 | 1.4 | 1.2 | 61.9 | 1.3 | 1.4 |
| protein | | | | | | | | | | |
| LHCA3 | 100 | 100.0 | 0.0 | 0.0 | 57.5 | 4.4 | 3.4 | 48.5 | 2.1 | 1.5 |
| protein | | | | | | | | | | |
| LHCA4 | 100 | 100.0 | 0.0 | 0.0 | 56.6 | 2.6 | 1.7 | 42.9 | 1.3 | 1.2 |
| protein | | | | | | | | | | |

^{a}Includes leaf, stem, seedling, green shoot
^{b}Semiquantitative score
EB = embryo
IM = immature seed

Based on the above information the AGPase AGPS2a subunit was operably linked to the OsLHCA3 promoter (SEQ ID NO: 9), OsLHCA3 first exon (SEQ ID NO: 13), first intron (SEQ ID NO: 14), OsLHCA3 second exon (SEQ ID NO: 15), OSLHCA3 second intron (SEQ ID NO: 16) and OsLHCA3 terminator (SEQ ID NO: 17), after the cassette was modified to include a TMV-Ω translational enhancer (Gallie D R, Walbot, V. (1992) Nucleic Acids Res 20:4631-4638) and terminate in a soy-optimized Kozak sequence (SEQ ID NO: 18). The AGPase AGPL3 subunit was operably linked to the OsLHCA4 promoter (SEQ ID NO: 10), OsLHCA4 first exon (SEQ ID NO: 19), OsLHCA4 first intron (SEQ ID NO: 20) and OsLHCA4 terminator (SEQ ID NO: 21) after the vector is modified to include the TMV-Ω M15 sequence and terminate in a soy-optimized Kozak sequence (SEQ ID NO: 22). The FBPase was operably linked to the OsPSAK promoter (SEQ ID NO: 11), the OsPSAK first exon (SEQ ID NO: 23), the OsPSAK first intron (SEQ ID NO: 24) and OsPSAK terminator (SEQ ID NO: 25) after the vector was modified to include the NtADH translational enhancer and terminate in a soy optimized Kozak sequence (SEQ ID NO: 26). The SBPase was operably linked to the OsPSID promoter (SEQ ID NO: 13) and OsPSID terminator (SEQ ID NO: 27) after it was modified to include the TMV-Ω M14 sequence and a soy-optimized Kozak sequence (SEQ ID NO: 28).

The expression cassettes were sequentially ligated to a binary vector for agrobacterium-mediated transformation. This vector also includes a polyphenol oxidase expression cassette for plant selection (Li X, et. al. (2003) Plant Physiol 133:736-747). The trait gene ligation order from right border to left border was FBPase, SBPase and AGPase. An eFMV/e35S transcriptional enhancer complex is near the right border. This strategy enables coordinate expression of the four genes required for the trait. Each cassette is optimized for high protein expression.

### Example 3: Production of Transgenic Tobacco

The DNA of Example 2 was inserted into tobacco following standard agrobacterium-mediated plant transformation procedure (Li X, et. al. (2003) Plant Physiol 133:736-747). Based on primary and secondary TaqMan analysis, 42 single-copy, backbone free events were produced. Twenty-one of these events set seed. Transgene activity was assessed by qRT-PCR on T0 leaf samples. For each plant, the tip region of the youngest fully expanded leaf was sampled and transcript abundance relative to endogenous tobacco Alcohol Dehydrogenase 1 (ADH1) was determined for all four trait genes (Jian, B., et. al. (2008) BMC Mol Biol 9: 59). The average transcript abundance for each trait gene ranged from 1316 to 2978 in the fertile, single-copy, backbone free events. T0 events produced transcripts from all four trait genes between 0.5-fold and 5-fold of ADH1 levels, and the relative abundance was PSID:SBP> LHC3:AGPS>LHC4:AGPL>PSAK:FBA. It is notable that respective transcript levels among the 4 trait genes were highly consistent between events, and that position effect likely accounted for expression variation between events. The observed average increase in trait gene expression was close to the engineering objective. Based on T0 qRT-PCR data, one low (A123A), one medium (A148A), and three high expressers (A117A; A126A and A156A) were selected for further analysis.

### Example 4: T1 Segregation and T+ Expression Analysis

T1 seed for events A117A, A123A, A126A, A148A and A156A were surface sterilized and spread on plates containing Gamborg's B5 media plus 2% sucrose and either none, 100 nM or 200 nM butafenacil (Li X, et. al. (2003) Plant Physiol 133:736-747). The T1 seed germination ratios on Gamborg's B5 Gelzan plates containing the PPO herbicide (please see Table 4) were consistent with single insertions, and germination rates were between 82 and 97%.

TABLE 3

| | | | | | Butafenacil- |
|---|---|---|---|---|---|
| | | | Number of | | resistant T1 |
| Selection agent | | Number | established | Seedling | seedling frequency |
| (amount of | | of seeds | seedlings at | establishment | corrected for seed |
| butafenacil) | Event | sown | 8 dai* | frequency (%) | viability (%) |
| 0 nM | WT | 87 | 84 | 96.6 | |
| 0 nM | A117 | 57 | 51 | 89.5 | |
| 0 nM | A123 | 80 | 66 | 82.5 | |
| 0 nM | A126 | 85 | 83 | 97.6 | |
| 0 nM | A148 | 96 | 89 | 92.7 | |
| 0 nM | A156 | 109 | 101 | 92.7 | |
| 100 nM | WT | 68 | 1 | 1.5 | 1.5 |
| 100 nM | A117 | 78 | 48 | 61.5 | 69 |
| 100 nM | A123 | 71 | 48 | 67.6 | 82 |
| 100 nM | A126 | 95 | 77 | 81.1 | 83 |
| 100 nM | A148 | 81 | 62 | 76.5 | 83 |
| 100 nM | A156 | 93 | 53 | 57.0 | 62 |
| 200 nM | WT | 77 | 0 | 0.0 | 0 |
| 200 nM | A117 | 55 | 36 | 65.5 | 68 |
| 200 nM | A123 | 94 | 64 | 68.1 | 76 |
| 200 nM | A126 | 93 | 68 | 73.1 | 89 |
| 200 nM | A148 | 86 | 60 | 69.8 | 71 |
| 200 nM | A156 | 74 | 53 | 71.6 | 77 |

*Days after imbibing.

Trait gene transcription was robust in T1 plants but did not correlate well with T0 generation data. Of the five events chosen for T1 analysis, three events (A117A, A126A and A156A) were selected for in-depth physiological analyses at the T2 generation. Trait gene activity was detected across three generations.

### Example 5: Trait Temporal Expression and Transcript Processing

These events were further analyzed by looking at the (i) the circadian transcript abundance of each trait gene and (ii) the pre-mRNA splicing efficiency of intron-containing trait transcripts.

The regulatory sequence used to express the three genes was active in green tissue and was light regulated. Transcript abundance should peak early to mid-afternoon. Transcript levels were measured for each trait gene by qRT-PCR in leaf samples collected every 4 hours, over a 24 hour period. The transcript levels were low during the night and increased from dawn to a peak at around 1500. Afterwards transcript levels declined. This suggests that the rice regulatory sequences functioned as expected in transgenic tobacco. Three of the four trait gene expression cassettes (PsAK:FBPase; LHC3:AGPSS; and LHC4:AGPLS), produced disrupted transcripts. The introns used in the expression cassettes were derived from rice sequences. To determine if the rice (monocot) intron donor-acceptor (GT-AG) sites are recognized and the efficiently spliced in tobacco cells (dicot) (Hanley, B. A., Schuler, M. A. (1988) Nucleic Acids Res 16: 7159-7176; Goodall, G. J., Filipowicz, W. (1991) EMBO J 10: 2635-2644), the first strand cDNA was synthesized by reverse transcription of mRNA, and primers from the flanking introns were used to detect their presence by PCR. The PCR products were also sequenced to confirm the agarose gel electrophoresis results. PCR products corresponding to the expected spliced transcript were recovered for all three trait genes in two different events. The PsaK:FBPase and LHC4:AGPLS trait genes produce a significant proportion (between 30% and 50%) of unspliced transcript. An unspliced version of the LHC3:AGPSS transcript was not detected, but approximately 30 to 40% was a mis-spliced form that contained 48 additional nucleotides upstream of the expected AG donor site was identified. Such mis-splicing of monocot introns in dicot systems is consistent with previous reports (Hanley, B. A., Schuler, M. A. (1988) Nucleic Acids Res 16: 7159-7176; Goodall, G. J., Filipowicz, W. (1991) EMBO J 10: 2635-2644). Together the data indicate that the rice introns were recognized, but may not be efficiently processed in tobacco. Despite this observation a significant portion (between 50 and 70%) of trait transcripts were present in the correct, mature forms indicating that the trait is functional at the molecular genetic level.

### Example 6: Fructose 1,6-Bisphosphate Aldolase Activity in Transgenic Tobacco Leaf Tissue

Tips from the youngest fully expanded leaf of homozygous trait positive and null plants were sampled. Protein extracts were prepared from 130-180 mg fresh weight tissue using a common extraction buffer (Iwaki, T., et. al. (1991) Plant Cell Physiol 32: 1083-1091; Muller-Rober, B., et. al. (1992) EMBO J 11: 1229-1238; Harrison, E. P., et. al. (1998) Planta 204: 27-36). To quantify aldolase activity, the rate of FBP cleavage was followed by oxidation of NADH via absorbance at 340 nm in a coupled-enzyme assay. To reduce biological variation when comparing homozygous trait positive and null plants, consistent plant growth conditions (illumination, irrigation, fertilization and similar plant orientation) were maintained. To reduce technical variation during sample preparation and the activity assay, null and trait positive plants samples were alternatively processed.

FBP aldolase activity in homozygous trait positive and null T2 plant leaf extracts could not be distinguished between the trait positive and null plants. Despite high coefficients of variation (CV) in this assay, we found very similar total aldolase activity between homozygous and null plants. A possible explanation is the assay's inability to distinguish cytosolic and plastidial FBP isoforms, although some reports indicate the plastidal isoform accounts for up to 90% of cellular activity (Haake et al., (1999) Plant J 17: 479; Miyagawa et al., (2001) Nature Biotech 19: 965; Lefebvre et al., (2005) Plant Physiol 138: 451; Smidansky et al., (2007) Planta 225: 965)

### Example 7: Analysis of Photosynthetic Apparatus

Several experiments to physiologically assess the engineered trait were conducted. The purpose was to determine if homozygous trait positive plants had distinct photosynthetic properties compared to null plants. To ensure detection of trait effects in homozygous trait positive plants, when compared to null plants, leaf samples (for protein quantification and biochemical assays) and physiological experiments were conducted between 1400 and 1600 hours.

Chlorophyll fluorescence was measured in young plants (leaves 1 to 4) then CO₂ assimilation rates were measured in the youngest fully developed leaf (leaves 5 to 7) of more mature tobacco plants. These experiments included 12 homozygous trait positive and 12 null plants from the T2 generation of events A117A, A126A and A156A.

Chlorophyll fluorescence was measured as a diagnostic for in vivo photosynthetic activity (Baker, N. R. (2008) Annu Rev Plant Biol 59: 89-113). Leaves on 3-4 weeks old tobacco plants at growth stages 12 to 17 (Lancashier, P. D., et. al. (1991) Ann. appl. Biol. 119: 561-601 are flat and horizontally oriented to the light source.

Using the theory of chlorophyll fluorescence measurements (CFM), Fq′/Fm′ was calculated, which provides a diagnostic of PSII operating efficiency. This estimates the linear electron transport rate, thus the NADH and ATP consumption rate, thus the RuBP regeneration rate. This is a good indicator of changes in the quantum yield of CO₂ assimilation (Baker, 2008) and was previously shown to correlate with increased SBPase activity and the CO₂ assimilation rate in young tobacco leaves (Lefebvre, S., et. al. (2005) Plant Physiol. 138: 451-460). The Fv/Fm was also calculated, which represents the PSII maximum quantum efficiency. Table 4 shows that the homozygous trait positive plants were not significantly different from null plants.

Table 4a and b. In vivo photosynthetic activity of young plants leaves assayed by chlorophyll fluorescence. T2 progeny of A117A-10 (null), A117A-11 (hom), A126-1 (null) and A126-5 (hom) T1 plants were analyzed. No significant differences (Students t-test p<0.05) were found. Data are the mean±SD (n=12). Note that ‘horn’ is ‘homozygous trait positive’.

TABLE 4a

| | | leaf #1 | | leaf #2 | |
|---|---|---|---|---|---|
| | genotype | A117 | A126 | A117 | A126 |
| PSII operating | null | 0.585 ± 0.010 | 0.610 ± 0.016 | 0.565 ± 0.013 | 0.593 ± 0.027 |
| efficiency | hom | 0.575 ± 0.018 | 0.611 ± 0.019 | 0.554 ± 0.021 | 0.584 ± 0.027 |
| (Fq′/Fm′) | | | | | |
| PSII maximum | null | ND | ND | ND | ND |
| quantum efficiency | hom | ND | ND | ND | ND |
| (Fv/Fm) | | | | | |

TABLE 4b

| | | leaf #3 | | leaf #4 | |
|---|---|---|---|---|---|
| | genotype | A117 | A126 | A117 | A126 |
| PSII operating | null | 0.546 ± 0.014 | 0.560 ± 0.018 | 0.546 ± 0.014 | 0.560 ± 0.018 |
| efficiency | hom | 0.549 ± 0.021 | 0.548 ± 0.028 | 0.549 ± 0.021 | 0.548 ± 0.028 |
| (Fq′/Fm′) | | | | | |
| PSII maximum | null | 0.779 ± 0.009 | 0.795 ± 0.009 | 0.779 ± 0.009 | 0.795 ± 0.009 |
| quantum efficiency | hom | 0.773 ± 0.016 | 0.795 ± 0.010 | 0.773 ± 0.016 | 0.795 ± 0.010 |
| (Fv/Fm) | | | | | |

The CO₂ photoassimilation rate was assayed on 2.5 cm² source leaf patches in older plants by infra-red gas analysis (IRGA). The CIRAS-2 IRGA device was fixed to a tripod to gently clamp the gas exchange cuvette to leaves and minimize data noise generated by plant handling. The environment applied to the leaf patch was programmed to mimic the growth chamber environment (400 μmol moF¹ CO₂; 26° C.; ambient humidity) to assess steady-state photosynthesis under standard growth conditions. The initial analysis examined the youngest fully expanded leaf of homozygous trait positive and null T1 plants (4<n<6). There was no significant difference in photoassimilation between homozygous trait positive and null plants. Measurements were then taken from a larger population of T2 plants. In addition, plants subjected to sub-optimal growth temperatures for 18 hours (12° C. and 37° C.) prior to each measurement. Although temperature affects the observed photoassimilation rate, there was no significant difference between homozygous trait positive and null plants.

The CIRAS-2 IRGA system can vary CO₂ levels applied to the leaf patch from 10 to 1500 μmol·mol⁻¹. The photoassimilation (A) response to intracellular CO₂ (CO reports the in vivo regulation and limitation of photosynthetic activity. Specifically, at low Cᵢ (10-300 μmol mol⁻¹) rubisco catalytic activity is the limiting factor, and at intermediate Cᵢ (300-700 μmol mol⁻¹) and high (700-1300 μmol mol⁻¹) the RuBP regeneration rate and triose-phosphate utilization become rate-limiting, respectively.

SBP is a critical control point in RuBP regeneration, and several reports show that SBPase over-expression has a positive effect on photoassimilation (A) and plant growth (Miyagawa, Y., et. al. (2001) Nature Biotech 19: 965-969; Lefebvre, S., et. al. (2005) Plant Physiol. 138: 451-460). In addition, SBPase over-expression had the highest transcription among the four trait genes comprising this photosynthesis enhancement construct. Therefore, A/C, curves were constructed to determine how the trait effects RuBP regeneration.

A total of 15 A/Cᵢ curves, 7 null and 8 homozygous trait positive, were built using T1 plants representing 5 selected events. The data clearly showed no significant difference between homozygous trait positive plants and null plants. There was a slight but insignificant decrease in trait positive photoassimilation (A) at intermediate Cᵢ levels corresponding to the RuBP regeneration limiting phase.

### Example 8: Plant Growth Assessment

Various plant growth parameters were measured to assess the effect of trait gene expression on plant growth and development. These included leaf chlorophyll content (SPAD meter value), leaf size (length and width) and plant shoot height.

Harrison and co-workers found reduced chlorophyll content in SBPase-antisense tobacco plants, indicating that SBPase influences chlorophyll content (Harrison, E. P., et. al. (1998) Planta 204: 27-36). Chlorophyll content was measured at the tip of the youngest fully expanded leaf using a SPAD meter. T1 plants representing 5 events were assayed, and there is no significant difference in chlorophyll content between trait positive and null plants. Although SPAD meter data are not as robust as direct chlorophyll extraction/quantification (Harrison, E. P., et. al. (1998) Planta 204: 27-36), the assay is non-destructive and provides a good first approximation. In addition, the data show that plant growth conditions (soil, irrigation, nutrition, light, temperature etc.) were highly homogenous.

The size of the youngest fully expanded leaf and plant height were measured with a ruler. Some significant differences in leaf properties between trait positive and null plants for events A123A, A126A and A156A were observed, but the pattern was not consistent and the sample size was small. No significant differences in plant shoot height were observed between trait positive and null plants. The low data variance indicates a very high level of plant homogeneity in this growth environment (e.g. the fully developed leaf #6 at 35 days).

Although the differences between homozygous trait positive and null plants were very small and mainly non-significant, small differences in photoassimilation rates may produce a significant effect on overall plant biomass accumulation over 45 days of growth. To test this, shoot biomass was measured (grams DW) in T1 and T2 plants. T1 A126A trait positive plants have a small, but significant increase in shoot biomass, but this was not observed for other events. Furthermore, no significant difference between A126A trait positive and null T2 plants were observed, and plant biomass was surprisingly consistent among the T2 plants.

### Example 9: Closed Chamber Monitoring of Whole-Plant Gas Exchange

It may not be possible to detect small, but significant changes in photoassimilation using an IRGA device An addition test was devised using large hypobaric chambers (Wheeler, R. M., et. al. (2011) Adv Space Res 47:1600-1607) to monitor with high precision plant CO₂ demand, night time respiration and transpiration of a 29 plant population throughout development. Event A126A was chosen for these experiments because, among all the measurements performed, this event presented some significant differences between homozygous trait positive and null plants in leaf size and shoot biomass. The data in this study were collected on a chamber basis, one chamber contained A126A-5 (homozygous trait positive) T2 plants and a second chamber contained the of A126A-1 (null) T2 plants. The experiment was repeated twice.

The study phases were germination, thinning, growth, response to environment and maturation. Excess seed were germinated for each chamber to ensure establishment of a uniform population. The plants were thinned to 30 per chamber, after which the chambers were sealed for the duration of the study. At the end of the study 29 plants developed in each chamber. Several plant growth-related chamber parameters were monitored during the study including atmospheric CO₂ and O₂, CO₂ demand to maintain a 400 ppm set point and condensate. The CO₂ data were used to calculate two photosynthetic rates. The first is CO₂ draw down that occurs at the beginning of the light period, in which the CO₂ released during the dark period is reacquired. The second is steady state photosynthesis, in which the CO₂ required to maintain an [CO₂]ₐₜₘ of 400 ppm. The CO₂ data were also used to calculate the night time respiration, by monitoring CO₂ released during the dark period. The condensate data were used to calculate daily transpiration rates. The mean condensate data collected during the germination period was used to establish instrument background, for the daily transpiration rate calculations. Both a dark period and light period transpiration rate was calculated. The daily steady state photosynthetic and transpiration rates were used to calculate daily water use efficiency.

The general trend in both replicates is that the null plants out-performed the homozygous trait positive plants in terms of daily net CO₂ assimilated, daily CO₂ assimilation rates and night time respiration rates. Plant response to change in both CO₂ level and temperature were examined. Homozygous trait positive and null plants responded similarly to the environmental perturbations. This contributed to the increased biomass produced by the null plants relative to the homozygous trait positive plants. Table 5 shows that across both replications the nulls produced approximately 30% more aerial biomass, or about 4 kg. Taken together with data presented in previous examples, a general conclusion is that the trait does not work. Plant photoassimilation by all measures was lower in trait positive plants, relative to null plants. Although there was no observed difference in photoassimilation observed, Table 5 shows the number of developing reproductive structures was significantly increased by the end of the study. In both replications the homozygous trait positive plants produced significantly more healthy seed pods than the null plants. In replication 1 (chambers 2 & 3), the difference was more than 3:1 and in replication 2 (chambers 4 & 5) the trait positive plants produced˜72% more pods. In replication 1, an unexplained ethylene spike in the null chamber at the transition to reproductive development likely caused significant pod abortion. This was not observed in replication 2, and ethylene eventually returned to comparable levels in replication 1. Nevertheless, the difference in pod set between the transgenics and nulls was highly significant. Although not to be limited by theory, this observation suggests that the simultaneous overexpression of an FBPase, and SBPase and the large and small subunit of an AGPase resulted in greater partitioning of photoassimilate to reproductive structures, rather than increased photoassimilation itself.

TABLE 5
Summary of biomass production in Precision Chambers. Data are the mean ± SD (n = 29)

| | Fresh weight | Dry weight | DW:FW | Seed pods | Seed pods | Seed pods |
|---|---|---|---|---|---|---|
| Chamber | (g) | (g) | ratio | per plant | per gram FW | per gram DW |
| HC-2 | 588.5 ± 286.4 | 55.0 ± 27.9 | 0.096 ± 0.020 | 19.9 ± 18.7 | 0.027 ± 0.021 | 0.285 ± 0.224 |
| HC-3 | 442.3 ± 244.6 | 50.2 ± 28.5 | 0.113 ± 0.011 | 70.8 ± 48.3 | 0.184 ± 0.154 | 1.583 ± 1.244 |
| p-value | 0.04118063 | 0.51865947 | 0.00020546 | 0.00000204 | 0.00000131 | 0.00000088 |
| (t-test) | | | | | | |
| HC-4 | 619.3 ± 214.2 | 58.1 ± 22.8 | 0.094 ± 0.014 | 34.9 ± 26.7 | 0.052 ± 0.033 | 0.548 ± 0.323 |
| HC-5 | 479.5 ± 243.9 | 51.9 ± 26.6 | 0.111 ± 0.019 | 60.2 ± 42.4 | 0.112 ± 0.062 | 1.01 ± 0.487 |
| p-value | 0.0239980 | 0.3488579 | 0.0002618 | 0.0086275 | 0.0000264 | 0.0000792 |
| (t-test) | | | | | | |

Seed pod production on an aerial biomass basis was examined. Comparing aerial biomass on a plant basis did not distinguish homozygous trait positive from null plants. However trait positive plants had a significantly higher dry weight:fresh weight ratio. The senesced leaves were collected and weighed in each chamber. The homozygous trait positive plants shed significantly more leaf biomass than the null plants, indicating that the homozygous trait positive plants had an early leaf senescence phenotype. The difference in number of seed pods per plant, alone or as a function of aerial biomass, was statistically significant.

Early leaf senescence between the homozygous trait positive and null plants was not observed in initial growth chamber and greenhouse work. Possible explanations are that the growth chamber and greenhouse grown plants were managed in individual pots, which constrained growth and made it difficult to distinguish homozygous trait positive from null plants. In the closed chamber environment access to water and nutrients, including CO₂ was not limiting. However the plants produced a canopy and had to compete for access to available light. The plants also had to cope with increasing levels of oxygen.

### Example 10: Expression Cassette Performance in Monocots and Dicots

Several maize transformation vectors were constructed and used to test the expression cassettes found in the tobacco transformation vector. Table 6 outlines the components in each vector. There are subtle differences in sequence but the global expression control elements are derived from the same source gene. For example some promoters don't include a translational enhancer, since these tend to inhibit expression cassette activity in maize

Transgenic maize were generated using each binary vector in Table 6, and leaf tissue from primary transgenic plants, or the initial regenerants, was sampled for qRT-PCR analysis. Only single-copy, backbone-free events were analyzed. The results in Table 7 show that all four expression cassettes are transcriptionally active in tobacco and maize. The activity level varies between constructs, and the maize variation is likely due to the coding sequence. The data show that these expression tools are effective in both maize and tobacco.

TABLE 6
Binary vectors used to evaluate light regulated expression cassettes from rice. Each expression
cassette consists of a promoter and a terminator. The suffix indicates version number.

| | transformation vector | | | | |
|---|---|---|---|---|---|
| | 20023 | 18520 | 18284 | 17581 | 18994 |
| | transformed plant | | | | |
| source | maize | tobacco | maize | maize | maize |
| gene | expression cassette components | | | | |
| OsPSID | prOsPSID- | prOsPSID- | | | |
| | 01/tOsPSID-01 | 02/tOsPSID-01 | | | |
| OsPSAK | prOsPsaK- | prOsPsaK- | | | |
| | 01/tOsPsaK-01 | 02/tOsPsaK-01 | | | |
| OsLHCA3 | prOsLHC3- | prOsLHC3- | prOsLHC3- | | |
| | 02/tOsLHC3-01 | 03/tOsLHC3-01 | 03/tOsLHC3-01 | | |
| OsLCHA4 | prOsLHC4- | prOsLHC4- | prOsLHC4- | prOsLHC4- | prOsLHC4- |
| | 01/tOsLHC4-01 | 01/tOsLHC4-01 | 02/tOsLHC4-01 | 01/tOsLHC4-01 | 01/tOsLHC4-01 |

TABLE 7
Performance of light regulated expression cassettes in transgenic plants. Various trait genes were
used to generate qRT-PCR data. The data are reported as the ratio of the signal from the trait gene
and the signal from an endogenous control gene multiplied by 1000. In tobacco the endogenous control
gene is alcohol dehydrogenase and in maize the endogenous control gene is EF1-alpha.

| | | transformation vector | | | | |
|---|---|---|---|---|---|---|
| | | 20023 | 18520 | 18284 | 17581 | 18994 |
| | | transformed plant | | | | |
| Expression | | maize | tobacco | maize | maize | maize |
| cassette | generation | mean relative expression level (n = number of events) | | | | |
| OsPSID | T0 | 727 (n = 22) | 2978 (n = 19) | | | |
| | T1 | 2377 (n = 20) | 5011 (n = 3) | | | |
| OsPSAK | T0 | 486 (n = 22) | 1316 (n = 19) | | | |
| | T1 | 1166 (n = 20) | 16346 (n = 3) | | | |
| OsLHCA3 | T0 | 13486 (n = 22) | 1591 (n = 19) | | | |
| | T1 | 41108 (n = 20) | 2977 (n = 3) | | | |
| OsLCHA4 | T0 | 958 (n = 22) | 1472 (n = 19) | 4800 (n = 28) | 847 (n = 25) | 1797 (n = 37) |
| | T1 | 2817 (n = 20) | 4282 (n-3) | | 2817 (n = 20) | 2930 (n = 11) |

All references cited herein, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

1. A nonnaturally occurring light inducible regulatory nucleic acid comprising:
a. a regulatory nucleic acid having at least 90 percent or greater sequence identity to a nucleic acid selected from the group consisting of SEQ ID NO: 9, 10, 11 and 12; or
b. a regulatory nucleic acid selected from the group consisting of SEQ ID NO: 9, 10, 11 and 12 or a functionally equivalent fragment thereof; or
c. a regulatory nucleic acid selected from the group consisting of SEQ ID NO: 9, 10, 11 and 12;
wherein said regulatory nucleic acid directs transcription of an operably linked polynucleotide in a plant.

2. The nucleic acid of claim 1, wherein said sequence identity is at least 95 percent or greater.

3. The nucleic acid of claim 1, wherein said sequence identity is at least 98 percent or greater.

4. (canceled)

5. (canceled)

6. (canceled)

7. The nucleic acid of claim 1, wherein the nucleic acid is operably linked to an exon and an intron.

8. The nucleic acid of claim 1, wherein the nucleic acid molecule is operably linked to a terminator.

9. The nucleic acid of claim 7, wherein the exon and intron are isolated from the same gene as the nonnaturally occurring regulatory nucleic acid.

10. The nucleic acid of claim 8, wherein the terminator is isolated from the same gene as the nonnaturally occurring regulatory nucleic acid.

11. An expression cassette comprising:
a. a first nucleic acid according to claim 1;
b. a second nucleic acid to be transcribed, wherein said first and second nucleic acids are heterologous to each other and are operably linked; and
c. a terminator operably linked 3′ to the nucleic acid to be transcribed.

12. The expression cassette of claim 11, wherein the second nucleic acid is selected from the group comprising a pest resistance nucleic acid, a disease resistance nucleic acid, an herbicide resistance acid, a value-added trait nucleic acid, a photoassimilation regulated nucleic acid, a yield nucleic acid and a stress tolerant nucleic acid.

13. The expression cassette of claim 11, wherein the heterologous coding region is expressed in green tissue and light regulated.

14. The expression cassette of claim 11, wherein the first nucleic acid is operably linked to an exon and an intron.

15. A plant, plant tissue, or plant cell comprising the expression cassette of claim 11.

16. The plant, plant tissue, or plant cell of claim 15, wherein the plant, plant tissue, or plant cell is a monocot or from a monocot.

17. The plant, plant tissue, or plant cell of claim 15, wherein the plant, plant tissue, or plant cell is maize or from maize.

18. (canceled)

19. (canceled)

20. A method of transcribing a heterologous coding region in a plant, plant tissue or plant cell comprising:
a. providing the nonnaturally occurring light inducible nucleic acid of claim 1 operably linked to a heterologous coding region;
b. creating a plant, plant tissue, or plant cell comprising the nucleic acid; and
c. exposing the plant, plant tissue, or plant cell to light, in order to induce or activate transcription.

21. (canceled)

22. (canceled)

23. (canceled)

24. (canceled)

25. (canceled)

26. (canceled)

27. Use of a nonnaturally occurring nucleic acid to promote expression of a heterologous transgene in the presence of light, wherein the nucleic acid is selected from a group consisting of SEQ ID NO: 9, 10, 11 and 12.


SEQUENCE LISTING (28 sequences)

SEQ ID NO: 1 - DNA, 1757 bp, Oryza sativa
atatcccttttgcccttaaatctgagaagttgctgcacttctcaccccccctctaaggaaagactgcaaagaaaaggcctgtgtcaaggagagggtagtagcaccttgctctcctttgccttcagagaaaccggaagcaatggcgtctgctactctcctcaagtcatctttccttcccaagaagtctgaatggggtgccacccgccaggctgccgcccccaagccggtgactgtctccatggttgtccgcgctggtgcctacgacgatgagcttgtcaagaccgcgaaaaccattgcatcaccaggaaggggtatccttgccatggatgagtcgaacgcgacctgcggtaagaggcttgcgtcaattggccttgagaacaccgaggccaaccgccaggcttaccggaccctccttgtcaccgcaccgggcttgggacagtacatctccggtgctatcctcttcgaggagactctgtaccagtcaactgtagatggcaagaagattgtcgacatcctcactgagcagaaaatcgttccaggtatcaaggtcgacaagggtcttgtgccccttgctggctccaacaacgagtcatggtgccaaggtctcgacggccttgcctcgcgcgaggcggcatactaccagcagggcgctcgcttcgccaagtggcgcactgttgtcagcatccccaacggcccatctgaactcgccgtgaaggaggctgcctggggccttgcccgctacgccgccatttctcaggacaacgggctggtgccgattgtcgagcctgagatcctcctcgacggtgagcatggcatcgacaggaccttcgaggtggcgcagaaggtgtgggcggagaccttcttctacatggccgagaacaatgtgatgttcgagggcatcctcctcaagccaagcatggtgacacccggtgccgagtgcaaggacagggccacccctgagcaagtatctgactacaccctcaagctcctccacagaaggatcccccctgccgtccccggcatcatgttcttgtcgggtgggcagtcggaggtggaggcgacgcagaacctgaacgcgatgaaccaggggcccaacccgtggcacgtgtcgttctcgtacgcgagggcgctgcagaacacgtgcctcaagacgtggggcgggcagccggagaacgtgaaggcggcgcaggacgcgctgctcctccgcgccaaggccaactcgctggcgcagctcggcaagtacaccagcgacggcgaggccgccgaggccaaggagggcatgttcgtcaagaactacgtctactaatccgatccaatccaatcaagacagtgaacctgctgctgctgctgctacgtactacagctactgctatactgctagtacctcgagggcactgtagccagtgtgtcgtcacagttacaggttgagatcgatggacaaagttaaaactatactgcattaattaattttaccttgagaacatacgtggttcatcatgtatttcatcacatgaatcttttctagagcgtttgatatacatacctcgtgttttgcctctgcaataaccttgtgtgtagacgaatctgtatactacctgtatgtgtggcctgtgaatcgatttggttactgcaaagttttgcgtatatatacatgcttgtgattctggaattattctggttacccccttagccttgcttcacaatctgttccttgatggggcaccacataaattgaagcaaaaattgtctcataaaag

SEQ ID NO: 2 - protein, 388 aa, Oryza sativa
MASATLLKSSFLPKKSEWGATRQAAAPKPVTVSMVVRAGAYDDELVKTAKTIASPGRGILAMDESNATCGKRLASIGLENTEANRQAYRTLLVTAPGLGQYISGAILFEETLYQSTVDGKKIVDILTEQKIVPGIKVDKGLVPLAGSNNESWCQGLDGLASREAAYYQQGARFAKWRTVVSIPNGPSELAVKEAAWGLARYAAISQDNGLVPIVEPEILLDGEHGIDRTFEVAQKVWAETFFYMAENNVMFEGILLKPSMVTPGAECKDRATPEQVSDYTLKLLHRRIPPAVPGIMFLSGGQSEVEATQNLNAMNQGPNPWHVSFSYARALQNTCLKTWGGQPENVKAAQDALLLRAKANSLAQLGKYTSDGEAAEAKEGMFVKNYVY

SEQ ID NO: 3 - DNA, 1698 bp, Oryza sativa
gcacgacttatctggtccagtcccagtgtcctcccaaatccgcgagcccgggcttggtgcaccccgcagcagctttgcctggcaacgtaaaaacgtactacggaaacttgtacactatcaagctcagcaactgcctgcccctctctttccctcgccattaagttttctcgtccggcggcgtcacccctgcgaattaactaactagctaggccggccatggagacggtggccgcggccagctacacccgcggggcggcgacgcgctcgccagcgtgctgcgccgccatgtccttctcgcagtcctacaggcccaaggccgctagaccgccgagcacgttctacggcgagtcgctgcgggttaacacggcgaggtcgctgccgtctgggaggcagtccaaggcggcgagccgggcggcgctcagcacccggtgcgagatcggggacagcctggaggagttcctgaccaaggcgacgccggataagaacctcatcaggctgctcatctgcatgggggaggcgatgaggacgatctcgttcaaggtcaggacggcgtcgtgcggcggcaccgcctgcgtcaactccttcggtgacgagcagctcgccgtcgatatgctcgccgacaagctcctcttcgaggctctggaatactctcatgtctgcaagtatgcgtgctccgaggaagtcccggagctgcaagacatgggtggaccagtcgatggtggtttcagtgtagcattcgaccctcttgatgggtccagcattgtcgacacgaacttcacagttggaaccatatttggtgtctggcctggtgacaagctgacaggcgtcaccggtggagaccaagttgctgctgccatgggcatctatggccctcgcactacttacattatcgctctcaaagattgtcctggaactcatgaattccttcttcttgatgaaggaaaatggcagcatgtcaaggacaccacaaccattggagaggggaaaatgttctctcctggtaacctgagggctacatttgataatcctgaatatgacaagctcatcaactactatgtcaaggagaagtacacattgcgttacactggaggaatggttcctgatgtcaaccagatcatagttaaggagaagggcatcttcaccaacgtgacgtctccgacagccaaggccaagctgaggctcctgttcgaggtggcgccgttgggtttcttgatagagaaagccggcgggtacagcagcgacggcaaacagtcggtgctggacaaggtgatcaacaacctcgacgagcggactcaagtggcctacggttccaagaacgagatcatccggttcgaggagactctctacggctcgtcgaggctcaccgccggcgccaccgtgggcgccgccgcctaatcttttgctgcaacgcgagcccagtgtctctgcaggcctcaaaaacgctgaaactgcagtcgttttagactcccatcctgtaatgcagattgggttgtagttgaagtgggacttgaaactgcaaagttatactcctttttgcaaggtacccgtttttttccccttttggtgtaaacagttgcatataaaggtaaattcgcatttctgttttgccactaagcaacacatcaacgagagcagcaacagcgagcaacagaatgtgcgaactatatcataatcgagatggacatgttgtgcttccaa

SEQ ID NO: 4 - protein, 392 aa, Oryza sativa
METVAAASYTRGAATRSPACCAAMSFSQSYRPKAARPPSTFYGESLRVNTARSLPSGRQSKAASRAALSTRCEIGDSLEEFLTKATPDKNLIRLLICMGEAMRTISFKVRTASCGGTACVNSFGDEQLAVDMLADKLLFEALEYSHVCKYACSEEVPELQDMGGPVDGGFSVAFDPLDGSSIVDTNFTVGTIFGVWPGDKLTGVTGGDQVAAAMGIYGPRTTYIIALKDCPGTHEFLLLDEGKWQHVKDTTTIGEGKMFSPGNLRATFDNPEYDKLINYYVKEKYTLRYTGGMVPDVNQIIVKEKGIFTNVTSPTAKAKLRLLFEVAPLGFLIEKAGGYSSDGKQSVLDKVINNLDERTQVAYGSKNEIIRFEETLYGSSRLTAGATVGAAA

SEQ ID NO: 5 - DNA, 1930 bp, Oryza sativa
gaagctaccccactagacaatctgtccactcaccactggcctcacttccttgatctcttctcgtcttctccaccccgcacgcggccacccccgcagggaccccgtgacccgcgcccgcgcccgcgcctcaccgcaatggcgatggcggcagccatgggcgtggcctccccctaccacgccgcccacgccgccgcatccacatcgtgcgactcgctccgcctcctcgtcgcggaaggccggccgcggcggccgcgaggggtggcctcctcctcctcctcctcctcttcggcggggaggcggaggcggccgctcgtcttctccccgcgcgcggtgtccgactccaagagctcgcagacctgcctcgacccggacgccagcacgagtgtgcttggaatcattcttggaggtggtgcagggactagattgtatcccctcaccaagaagcgtgccaagcctgccgtgcctctgggtgccaactacaggcttattgatatccctgtcagcaactgtttgaacagcaacatatccaagatctatgtgctgacacaattcaactctgcctctctgaaccgtcacctgtcaagagcctatgggaacaacattggcgggtacaagaatgaagggttcgttgaagtcctcgctgcacagcagagcccagataatcctaactggtttcagggtactgcagatgctgtaagacagtacttatggctatttgaggagcataatgttatggagtttctaattctggctggagatcacctttaccgcatggactatgaaaagttcattcaggcacacagagaaacagattctgatattactgttgctgccctgccaatggatgagaaacgtgcaactgcattcggcctcatgaaaattgacgaggaagggagaatagttgaatttgcagagaaaccaaaaggagaacaattgaaagcaatgatggttgacactaccatacttggccttgacgacgtgagggcaaaagaaatgccttatattgctagcatgggtatctatgtgattagtaaaaatgtaatgcttcagctcctccgtgaacaatttcctggagctaatgactttggaagtgaagtcattccaggtgcaacaaacatcggaatgagggtacaagcttacttgtatgatggttactgggaagacattggtaccattgaggcattctataatgcaaatctgggaataacgaaaaagcctgtaccagatttcagtttctatgaccggtctgctccaatttatacacaacctcgacacttgcctccttcgaaggttcttgatgctgatgtgacagacagtgtcattggtgaaggttgtgttattaaaaactgtaagatacaccattcagtagtgggactccggtcctgcatttctgaaggcgcaataatagaggactcattactcatgggagctgactactacgagactgaagcagacaagaaactccttggtgaaaaaggtggcattcccattggtattgggaagaattgccacattagaagggcaatcatcgacaagaatgctcgtattggagataatgtgaagataatcaatgttgacaatgtccaagaagctgcaagagagactgatggatactttatcaaaagtggcattgttaccgtgatcaaggatgctttgctccctagcggaacagttatatgaagtagctgcgtaatatgtgatggggcatcggcgacgagcaccaggcgggcattataagaagaataaaagcagttcaaggcactttattccctttttttttcttctgttcttcctctattgtgttaggataaatgaatggtagaagcaactgtttttcagatgttttgggagtgaatgataccgtgcttgaattttccatgttttgctttgcctctgcttgaaaatgaatccagcaatcccatgtgtttct

SEQ ID NO: 6 - protein, 514 aa, Oryza sativa
MAMAAAMGVASPYHAAHAAASTSCDSLRLLVAEGRPRRPRGVASSSSSSSSAGRRRRPLVFSPRAVSDSKSSQTCLDPDASTSVLGIILGGGAGTRLYPLTKKRAKPAVPLGANYRLIDIPVSNCLNSNISKIYVLTQFNSASLNRHLSRAYGNNIGGYKNEGFVEVLAAQQSPDNPNWFQGTADAVRQYLWLFEEHNVMEFLILAGDHLYRMDYEKFIQAHRETDSDITVAALPMDEKRATAFGLMKIDEEGRIVEFAEKPKGEQLKAMMVDTTILGLDDVRAKEMPYIASMGIYVISKNVMLQLLREQFPGANDFGSEVIPGATNIGMRVQAYLYDGYWEDIGTIEAFYNANLGITKKPVPDFSFYDRSAPIYTQPRHLPPSKVLDADVTDSVIGEGCVIKNCKIHHSVVGLRSCISEGAIIEDSLLMGADYYETEADKKLLGEKGGIPIGIGKNCHIRRAIIDKNARIGDNVKIINVDNVQEAARETDGYFIKSGIVTVIKDALLPSGTVI

SEQ ID NO: 7 - DNA, 2067 bp, Oryza sativa
ggtgtccctagtggaaagttgcacgagctcacgggggtcaggcgactaggcgaggagagaagaggagaggagaggagaggagaggtttggattgggaaggatcaccagctcaccacccgtttctctctccggcattgcgaggggaaggcggcgacgggaggcgcgcgcgggtgtagacgcgtagtgatggcggcgatggacctgcgggtggccgccccggcctccgtggccgcggcggcgcggtgcggcacgagcctggcgaggccgtggcctgcccgggcggtgggaggaggaggaggaggaggaggacggggccggcgtctctccgtgcggacgtccgtcgcgaccaccgaggccgccgccgccgccgtgggcgcctcggaggacgcggcgctggaggcgcgggactcgaagacggtggtggcggtcatcctcggcggcggcgcggggacgaggctgttcccgctcaccaagcgcagggccaagcccgcggtgcccattggcggagcgtacaggctgatcgacgtgccgatgagcaactgcataaacagcggcatcaacaaggtgtacattctcacccagttcaactccgcgtcgctcaaccggcacctttcccgggcgtataacttcagcaacggcgtcgcgtttggagatggattcgtcgaggtcctagcagcaactcagacgcctggatcggagggaaagaggtggtttcagggtacagctgatgctgttcggcaatttgactggctttttgatgatgccaaagctaaagacatcgatgatgtgctgattctttctggtgatcatctctaccgcatggactacatggactttgttcagagtcatcggcaaagaggtgcagacatcagcatctgttgcttgcctattgatgatagccgggcatctgattttggtctaatgaagatagacgacacaggaagagttatcgcatttagtgaaaaaccaaaaggagatgatttaaaggcaatgcaagttgacaccactgttctaggcttaccacaggatgaagcaaaggagaagccatacatagcgtcaatgggggtttatatatttaagaaagagatacttctaaatcttttgagatggcgttttcccacagcaaatgactttggatctgaaatcattcctgcttcagcaaaagaaatcaatgtaaaggcatatcttttcaatgattactgggaagacataggaactataaaatcattctttgaagccaatctttctcttgctgaacagccaccgagattcagcttttatgatgctaataaaccaatgtacacatcacggagaaacttaccgccatctatgatcaataatagtaagatcactgattcaatcatttcccatggatgtttcttggatagctgcaggatagagcacagtgttgttggaatccgttctaggataggttccaatgtgcacctcaaggatacagtaatgcttggtgctgatttctatgagactgatctggaaagaggagaactgctggctgaaggaaaggttcctattggaatcggggagaatacaaagattcagaactgcataatagacaagaatgcgaggatagggaagaatgtaacaatttccaactctgagggtgtacaggaagcagataggacctctgaaggcttctacatccgatcaggcatcaccatcgtattaaagaactcgataattgcagatggattagtcatctgaggtggaataaagccctcccttatccaaccagaaaaagccagccatgaccatttgcggcaggaatagaataaaatcatcagagctctttgttcttagttgtgtacttagcattcaagagcttctgcatgtctgtacaaaatcattgtctcgtgaatctaccgtgtgtgttttcacactaaacacatggtcatgaagctcgaaaaaaaaaacaaaattttatgatctggggtattggttgttccatgtaatctgcaggcgaagaaaaccttacctatctacaggtgtactctctccgtcaaaaaaaaaaaaacttaatctagatgggaatttgacacaaccttgaac

SEQ ID NO: 8 - protein, 511 aa, Oryza sativa
MAAMDLRVAAPASVAAAARCGTSLARPWPARAVGGGGGGGGRGRRLSVRTSVATTEAAAAAVGASEDAALEARDSKTVVAVILGGGAGTRLFPLTKRRAKPAVPIGGAYRLIDVPMSNCINSGINKVYILTQFNSASLNRHLSRAYNFSNGVAFGDGFVEVLAATQTPGSEGKRWFQGTADAVRQFDWLFDDAKAKDIDDVLILSGDHLYRMDYMDFVQSHRQRGADISICCLPIDDSRASDFGLMKIDDTGRVIAFSEKPKGDDLKAMQVDTTVLGLPQDEAKEKPYIASMGVYIFKKEILLNLLRWRFPTANDFGSEIIPASAKEINVKAYLFNDYWEDIGTIKSFFEANLSLAEQPPRFSFYDANKPMYTSRRNLPPSMINNSKITDSIISHGCFLDSCRIEHSVVGIRSRIGSNVHLKDTVMLGADFYETDLERGELLAEGKVPIGIGENTKIQNCIIDKNARIGKNVTISNSEGVQEADRTSEGFYIRSGITIVLKNSIIADGLVI

SEQ ID NO: 9 - DNA, 1547 bp, Oryza sativa
aggtacgtgggtccactggaatcaatgaaatatggtaccattgtgaaagctttatatcggttctcacgtaaatatcaaaattatggtaagtcttttttttttcatattggcagtttggcactgtatagagtagaaaattttctgccgttagccccttactgaaatgacttcagctatcgcaaaatgttatgtagcatttggcttttgttttgtaaaatgcttgcagaagaaaacatgatgtaaatattctcagtggctcatgttttagtcgctatatttctgctggccttgttcgataaatagacatatactccgtagaaggtgtttaactctcatgttctgaatgacaaatgttttgtccgcagcatttccgtaggctatgtttgccgtaatatggctgcattggaagttcttaattctaataccattttgtaggaggcatttcactcttggttattttccactacagtttcgatgtatttaactatcagttaaatcctcataaaagtacttcctccgtttcacaatgtaagtcattcttgcattttccacattcatattgatgtatctagacatatgtatctatctaaactcattaacatcaatatgaatgtggaaaaggctagaatgacttacattgtgaaacggagggagtagcattttttggctcttcatttgtacatagcgcttagcttgaatgattgataccaaccctttttatctaccaggtaaatgatactgagtctgagagcgaggtagtacttgtttacagaagacccatttttatgtgaaggggacgcttctggaactgaccaaatttcacgcaagtcttggcagctatcggcattgtgcttccttcctttctgatagagaatcgaaatgggctactcgtagaatgaatgttagtttgttttcgtcagaatgtaaaactacatatgtgcagttcagtggtaaaaactctcaaaccgaacacgtgtaccgagattgtcttgtttttcaccctgtgaaattcgctctcatctctcactgttgtacttgatgcacaaaatgtacaaactgccaagatattgtgtttttcaatgaatagtagttttacactgtttacttcctacatgacctgatgaccaaccatgactccatgatatattgtttccttttttcttttctttttttggttttaagattgattaatatctcatgtcatatcattttgtcaatttttttggctgaataactggcactagttccatcacaaatgcaggatagaaacaacaccaatctgtgctatccacaaagcctaaacaaaaaaatctcagcaaatgccaacaatccaagaacacaccatgcacagatgcaggaaatgcaaacctcagctgcatgctgtgctggcagctggtaagccactgagaggcagctcccttatctcacctgcccccctgccacgtggcacactcctactgggtccactgatgccctcatctgctaagatgccacctcacaccagaattagatttccccgggcccaccccttttccaaggccacaagc

SEQ ID NO: 10 - DNA, 994 bp, Oryza sativa
ggcttgtaactcgtctatgcagctattcatattgtaactcagtcagctccctgtcgcaaatgtgcctctgcgtcagtcgctgtctgtaaactgtccggcaattagaaattcccatccttagcatgcctggtattgttcagctcgaaactgaaatttttcttcgtgccctatattttttcggtgtagataagtgttccgctggaattttatgcaggtgctgtaccctatgtgctgctttttttttgtgtggggcgcccgccggggggggggggggggggggttgttacctggatgattgcaaataagaaccccggcaaatctgctggttggttgcaaataataacccctccaaatctgcgcagatgaaaccccattcaggacatgaattacgattgttcatgagctatttggatcatggaaagattggaaacaaactcttacgtcaaggtttctactaattacgtgattccgatttcagagtcagccttggctatactgcctttgctccagtaaacatcgctgctctagtaacaaacattgcagtaaacatcacaactatccaattcccttgttgctgctctagtaaaaaacattacaattatccaattcccagatattttttttcactgctccaaaacctgaagtacatatacgtgagttgagtgatccagcaacataaaaatccgaggctccgagcgatctgcaccaaccatctcacccgtccgacgtggcagcagcaaccagccacagctgagacctccatccaatagaaaccctccctttgattcccccgtatcccggcatccggataacgctggataagaggcgacgcctcccattggccacacccacccaacaacgcatcctggccgtccgatccacccccaccaccgatctccgccgtccgtcgccgccctcgccgccgtggacacctggcagcgccggccactcccggacagtttaatacaagccacgcctttgctccgtgccggcc

SEQ ID NO: 11 - DNA, 1728 bp, Oryza sativa
gtgcataaaatttgcattgaacaggttttgtgtgacaaggacagaacaccaaaaaaggtatttcctcctacatgtaatatattttttaaatcacaattcaccaggataataagcgttgcaatctacaagttacattcttttcttttctttttttggaaaaacaaacaatttaacaggcacaagtgatgggacatttggagcgaagaattttagaagtgaataggaagaggatcaaagttgtaaagcctggatccaaggcatcattcccaactacagaagtgcgaggaacatatgttccacccttccatgtaaattcttgcatgaacttggaaaataaaatgttcattttagaatgtataatccctgtcctacgcaaatttataactaaagctaactgttcattgaatctttcaggttgagctatatcgcaacgatcagcaccgcttcaagattgttgttgatggtgagagcgaagttgacttgatggtgcaaactcggcacatgcgagacgtcatcattctggtgattagaggtcttgctcaaaagttcaacagcacatctctcaactcactcctcaagatagaagcatgaagatcgagttgccaacctgatataacttacagtatgaaaccgattagaaaaggttgttcagtatcgattcaagttgtttagtttggaggatgtgtaatatttataggatttggagcgtggcagcttttgtgtatgggtgtggttgagtgtgttaaagtttttctttttctcaaatcaggagcgggttgctcttctgaccctggttttccatactcatgaaatgtacataggaaagggagggttggtgatatggtgttgcattaatatacaagttgtgtatgcaattctcatattaggctatatgattgattgggaagattttctgttttttttggctcgtcatcgttggtttcatatacaaaattcgaaatggaaatatgtacgagtgatggttattgaagtgtccattctattttaattctttgcgcgttgtgaggtaatctcatcgtggtgtaggcttacagcagtaagatgcgtcgccacgttgtttcacattgcaaaatccatttctgcaattgcaagcaatgggcttcaagttcttgattatttcagttcatgactcggcatagcaccagcaaaggagcaactggtaaagtggcacatccacttcagtgtcgatgcaagcaactcattccagcctacaggaacagtaaatccaggttcatttactcatttttctgtggttgaaaaataccaagatttccatttttcttactgctccacataattcctatttcccattctcttgcacttattccagcctacagggacagtaaatccaggttcatttactcatttttctgcggttgaaaaaataccaagatttccatttttttttactgctccacataattcctctttcccattctcttgctcactatatatgcctgtcaatttttttgacagaacatgcatcaagtcaccaccaacaacatttgcagctgcatccaagaacattgcatgcagaggtgagcatcttatccacattggcaggctctaaccaggagatgccacatcagcagcaaggatctgtaacctatccatgagtggccactgcagtccacaagctccccttgtggctcccattccacatctcctccaagaattaaagcccaatgcatctccaaggtggcactt

SEQ ID NO: 12 - DNA, 1918 bp, Oryza sativa
cttgagttttacatcattaaaattagcagtgtctttgtccaacaactttaaatatgtctgatacttctccatcctagtttgttccttctttgcttccgctgatttggtggttgcttctgtagatttcagcactgcttctgctctcaattttacagcttcgttgaggagaacaaaatcttgagatggctggtcccccaaaggagacggtgcatgtttttttcctttccctttaagcttgactttggcttttttctgcccttcaggacgcttctcttttcggctggtctcctcctcagtttcttggttggatgatgatgtatatgcaccagattcagaaatcttagtcctcttgttcttgctgtcctcttcaagtatccgtcgccattttggttgatcctttaattctctccacatatactctaatgtgaaagttttatcattgttttctgatttatataatgcatgggctttctccatgatcatatcatcagagaatccactgctccaggttcttctagcacgagaataagctccacaaaattttgcaatgtccttcttaacaccaccccaatgtgtcttacattgcacgactgtccttttgcggttattgctagatgtattaccattgaactctgcagcaacatccctccaatagtattctcccttcttgtcattgccatttatagaatctagtgagttattcaaccaagagctgaggagtcgtaagttttcttcttcggtccagtttgacgcgtgtccctcttctccctgtctcttggctattgtcacttgacttttcgatgttaaccaaattgttatcttcctgtcttgcagggcactgagaggctgatttggatccatgagatgaggcggacccgacaacttgatatggtgagtgcatccaactttctttaggatgaaaaccttgaaagcttattggtgcatattgtaaccagcttgcttgctactgaaaaccttgaaagtttggaggagaactaccatataggtgatagttggctggaggaccaaaagggttgaaggtttggaaattaggtggtgaggcttgggtaaaatgtggggatcggggaaatcgtgattgggagaaagttgaggggaattaagtaggaggggagttttggctagaatgttggcttggggagccttgttttaacaagttcatgaaacctctcttggagggatccatctcaggctctcagctagctaagtttcacaaaatgtgtttgtgttgatttaaagaaatcctgagccatctctatttttgtaggggatgaagatgataagaagaacagtccatgcaacggttggctagggaggagagcaacggttggaagcagcaatggctacttgcaatttaaaatttgttgtcagaactccactgtacacattctatttttttattccctttttaccggcagtagtgctacttgctggctttaactatgtaccacttccacaatagctaataccaggtagtaagaagccatttaatgcttactatctggtattttataacattgtgggtgccctaacaccaaccaggcagccacttttctagttttcttcctccaaccttgttgttcgatagccatgccatgagcgatggagcacctctctgccaccttgtagatctcctctactagccgtggcgtcagtgaggctgcagatggagcgctgcatgggtttgtggctgccatccacgagggtatagcgccgaagaacgacacactgacagatgccacatcagatagggagggaggggccagtgccacggtgacatgcatgtgtggaggccgcgtatcaaaatccctacccgccattggccaacctcatatccccttatccacttcctcacccatttcccctctcttcttcttactccatctccgatatctctctc

SEQ ID NO: 13 - DNA, 266 bp, Oryza sativa
acagcacccctgcaaccttgtgttcccctcatcccccttttctagcactcaccaagtttaatttggagcacacatttcagctcaccactgctagcttgggaagaggaggaggagcttgagcttggagagagacaacaatggcggctcaggctcttctctctgggaggcagctgctggggaggccattgcaatcttcagtctccaggtcttcttcctccaggaaggcacccttcatggtcagggcagaggccactccccctgccaag

SEQ ID NO: 14 - DNA, 97 bp, Oryza sativa
gtatatatgctgattgatttctactcttcctcaattcaaaattgaaagatattatgctgatctggttaaatttatgtttggtttggtttggtttcag

SEQ ID NO: 15 - DNA, 62 bp, Oryza sativa
caaggtgctgacaggcagctgtggttcgcatccaagcagtccctgagctacctggatggcac

SEQ ID NO: 16 - DNA, 548 bp, Oryza sativa
gtaagttttcatgtggtaaaatttattcatgttgcaataaaagaggcgttaaggcctcatttgaaacgattttcgaagaatttttataggaatcaaccgtaaaattcatgcgtttcaaaaaaaaaaaaaggagcttaattaagatggtaggaactagccgtatccaactgaatttcatagtacagtagtacaatcaaaatgatcaaacttttcataggaaacaataatcaatagaccttccaaataaataaataaataaagagtaaatttcagagaacctttacttttatggtcaaagatgacctgtggcacataaccgcatgatttatcgtctacggacctcatcttttcgtttatgcttatccttatcagcgaaactttaaatttttaatcttaaatttagagctaattttcatgttttttttatcgaagtttatttttcagcctttgcttctaaagtttttattcacaaattatatctcgtttgtaaatatgccaaggcttccataatattgatcgtgcgtgcatggaatcatggatggatgcag

SEQ ID NO: 17 - DNA, 1003 bp, Oryza sativa
agatgccatcaactacgtatttaattaattacgagtttttaattatgtatgtatgtacgtgcgtatgcgtatatgtgtgtacgtatgtatgtacgcaactttgtacgatcgatggtgcgtgtgatcgagaggggtatgtatgtacgtacgtatatacgatgtattattatgcatgggtgtagggtgtgggtgtaacatgtaaatttgtgctccaactgatttgcacgcatggtcgcatggatcgatttgctagcttgtctctcttttgttgggcatataactatttactacttttgttgattaaaagtttgtcactcaaagttccagtgataaatttttaattaacacttataaacgtggtaaatagttaaatatctcccttttattaggcggtagaactaggagtgtcccgtgtattttattttctccgtttttaaattattgtcatccacatgctacgctatgtaagatgtttcttagaagtatgtttatccctgacagtaaagaggaaaaaaatttttccggaagaaaactagaggagatttaattgcaccacataaaataatcttgtctagaaaaaaaaatctggtattccaaaagaaatagaaatataaaatcttgtggaccgtttggattggaatagtgatgaaggcctgagagatgctttactaggctggcccaaaattacatgatttggacatgggccacaacccacaaggcccatttttgaggtccacatgaaacaaacccacccattcccctcctcctctctcgagttccaaatgcgtcctctccgccgcacggccgcctccgcctccggcttcctccgccgccgcctcctctccgaggtcgccgggaacggcgcgccactcccgcgcgtcggggccgtgtacgccttcggggacaacagccacggcgcggcggggcagccggcgccggccgccgacgtctacgtccccacgcccgtgccctccctcccgacgtccgtcgccgccgtcgccgc

SEQ ID NO: 18 - DNA, 71 bp, Artificial Sequence
  Enhancer sequence
gttttacaacaattaccaacaacaacaaacaacaaacaacattacaattagtatttacataaaccaaaacc

SEQ ID NO: 19 - DNA, 250 bp, Oryza sativa
aaaacgtacccttgtgactacacccgcttcgcttcctcccctctctaagccggggaagctaagccaaggcgtccgtcaccgcccgcaccccggtcgcagccctccgctcgtcggcgtcgctcaagtctaccttcctagggcaatcctccacccgcctcgcccgcgcaccgactacgaggcgtattgttcgggcggaggccaagggagagtggctccccggcctcccttctcccacctacctcaacggcag

SEQ ID NO: 20 - DNA, 92 bp, Oryza sativa
gtgagcacacacataaaaaaaatatatttctactaccagtagagagcatcataaaaaaactactaacacgatagttaaacgggtgtgtacag

SEQ ID NO: 21 - DNA, 1159 bp, Oryza sativa
gcgtgtgatcgatttcatcagggccagggcatctcaaggagcttgatgagttcaggctggtgaaaccgatgattgggcgatggaagatgttctcttcttgtttcttctttttttttttgtggagtatgcatgtataagatgttaatgaattggggggaggagagagagagagatggatgtgatgagattcagacttactgtgtgtgttgtggtaattgtttcctgcatgcatggatctggatgcatgggtgagggggtgagttgagtggtgaatttctgatgtacagtactacagggggataaactatctcatggtagcagcagtgttctagctatctcatggtctcgatcttaattatggtggataaactacgcttaattgcttgtcaagtgcttcatttgcgcattgattcagtattgcgtatcgattcaaagacaaaaaaaaaaaaaaatccgtacagattcagtcacttttaatccagagctgcatccgcaatggtgaatgcttgctactctttggtgtattcccccaaaaaaatggtcaatgcgtacttatatttgttctgctaatacgcttaacaccagactcaccagtaaaagaatcacattttttttcaccacacaaaaaaagccataacaaaacaagataattctaataacgtttaagcttcttaataagatttactctgtttggtttcaacgcttcagagcagtcacacaaagcgatggtttgcaatgaacactttggatataatcagtaaaagaaatacctgcatgataatgcacacagatgcttcgaaaaccatcattagttattccttaacataatcacatatttctggactttacacataagctcgcagataattattctatcagtgataggctaggcaatgcatccgtcgacagcgagatgtctgcgatgacttagttaatcttaatatatgctccgctagtctttcaaaaatgctcacaaggatggtgtgtacgtgtgtgcgtttataggggtgagtatgcacaagttatgagagcttacagtttttttttaaaaaaaataagttcacagaatggaaaagatgatggttattttgacggaaattggccggccttcttcttcggctagcctggttaaagaaaagatcgactggtactacaaccg

SEQ ID NO: 22 - DNA, 79 bp, Artificial Sequence
  Enhancer sequence
tatttagtcgaacacaattacgtcgaacaacaacaaacaacaaacaacaaagtcgaacacaattacgtcgaccaaaacc

SEQ ID NO: 23 - DNA, 137 bp, Oryza sativa
gacattatttttttcctcgaaactcagctgcaaggagaaggagaaggtaggctccagccttggcttcccagctctccgccgccacatccgtgccgcagttccaaggcctccggacctactcctctcctaggtccttg

SEQ ID NO: 24 - DNA, 117 bp, Oryza sativa
gtcagttccttctatgatccaatgctatcaaattattttcctttgtaacgaaaagtataactagagatgcatacaattacacattcctaattgtccctggttcttgattaatcacag

SEQ ID NO: 25 - DNA, 987 bp, Oryza sativa
tcaaattcattaagctagtcacacgaatcaatactcctatgtgtaatcgatgcatgagtgcctgtgtgatggacgatgtaatgatcatgtaaattgtgcgtggaatcagtgcttatctagcttaattttgtttctgtcaaccaatggaatgtttcttgatgcagagctttgttgttggatcttcgaggaagaaaatgtttttgattgcttggttttaaagttctaaacacgaaaccttttcctcttaatgtttattttgtacatccctatgccattttttgtcacctgaaagttgtacatttttgcaactgcaaaattacatgagctttcacataggcttcaagaggcaagagctaattggtttctttctgctgagcaaatcctgattaatttggatactgaaagattaatcagcaaagatgaaaggaaagaggaggacaacatgcatgattccacgaaataaatcaacaagatactgaaataaacaaattttgggaggagtcagaattggaacgacattcagcagtgtccctagacactgcatgcacggatcctcatgactgggcgtcagtttagtatcgattcaagttgttagtttggaaatcaccaaagaagaaatgaaagaggagaacagcatgcaaaattccacgaaatataacaagataataaacaagagctgcaaaaagagatgaagtgcaaattttgggacgagtcagaattggaatgatattcagcagtgacacagtgtatctatcttcatcctcctccctcaaattaaccaatgttatttcatacaaaaactatctgacagaacaaaaatcacatcatcgagaaaaaagaattgcataacaactactgacaccaagaaaaaagagcagaccaaggagtagagaaagaaagtctaagggtaatacggaactttttctaatataaaagtaatggtaaaattaagctccaaaaatttgaacaagtcttatcgtaaacatc

SEQ ID NO: 26 - DNA, 88 bp, Artificial Sequence
  Enhancer sequence
atttaactcagtattcagaaacaacaaaagttcttctctacataaaattttcctattttagtgatcagtgaaggaaatcaagaaaacc

SEQ ID NO: 27 - DNA, 1022 bp, Oryza sativa
acttcttcttcttcttcttcttctctcataatcgacctattaattacttgtgttttgggtggatggacatgtaattttaatcatctgtcaggctgctcttcagttattgatgatggtgtaatcgatcgatggtgggatataatctcttcttataatattgtagtatatgtttggtgaattttgatgcgtgttcattaacgtgttaattattgtgacttaattcacatgatcaaacaatcgtaattcatcataattagttgctgcaagttaaattaattttccaacttttaggtagactaacactacacggctttattctggaaacgaatgctcccatcgatcatttggcttattcacggcatatttacaaatgaaaaacaatttgcgaatgaaactttagtatatatgttcttaacgatctaaaagcaaaaactgaaaaataaactttgatgaaagaaccccaaaactaactccaaatttaaggttgaaaattcaaattttgactaataatcataggaataagcgaaaaaacggagctaaaaattcagatatctgaacgtgagcaaatcacctctgatcattagcttcccaaccaatcagaaattctcaacatccatgtatggatatgtgctttgctattgattaagattgtgcatggttcacaatgattagataaaagaagtagaaaatgataagattaagataactaaacggaaattttattgttcatgcagtccccattgttccatgcatccagctaataatagccgtaaccgatccgttttttctcgttttttggattttttttttctcctcggataggtttacaggtgattcagttgcctcttcgtcatgacatattatcatctaataataagatatctaaagttcatccctcttcgcagagggaagaatcatacttgctgctatttggcttctcatcagcagagaccttgatgtcctgtggtttatcatcatccgagtcctttgtctgctcagcaactacatcagccttgtcatcg

SEQ ID NO: 28 - DNA, 75 bp, Artificial Sequence
  Enhancer sequence
gtttgtcgaacaacaacaaacaacaaacaacaaagtcgaacaacaacaaacaacaaacaacaaagtcgaccaaaa